(19) United States
(12) Patent Application Publication
(10) Pub. No.: US 2010/0298642 A1
(43) Pub. Date: Nov. 25, 2010

(54) MANIPULATABLE GUIDE SYSTEM AND METHODS FOR NATURAL ORIFICE TRANSLUMENAL ENDOSCOPIC SURGERY

(75) Inventors: Robert M. Trusty, Cincinnati, OH (US); Gregory J. Bakos, Mason, OH (US); Omar J. Vakharia, Cincinnati, OH (US)

Correspondence Address: K&L Gates LLP, 210 SIXTH AVENUE, PITTSBURGH, PA, 15222-2613, US

(73) Assignee: Ethicon Endo-Surgery, Inc., Cincinnati, OH (US)

(21) Appl. No.: 12/468,462

(22) Filed: May 19, 2009

(51) Int. Cl. A61B 1/01; A61B 1/005

(52) U.S. Cl. 600/114; 600/139

(57) ABSTRACT

A guide system for accommodating an endoscopic tool. The guide system comprises a flexible inner sheath and a handle coupled to the inner sheath adjacent a proximal end of the inner sheath. The inner sheath includes a plurality of working channels. The working channels are bundled over a common portion of their respective lengths, and the working channels collectively define a substantially honeycombed cross-sectional area.

### BACKGROUND

The embodiments relate, in general, to guide tubes for endoscopes and medical procedures and, more particularly, to devices for facilitating the insertion and manipulation of endoscopes and other surgical instruments within a body cavity to accomplish various surgical and therapeutic procedures.

Minimally invasive procedures are desirable because such procedures can reduce pain and provide relatively quick recovery times as compared with conventional open medical procedures. Many minimally invasive procedures are performed through one or more ports through the abdominal wall, commonly known as trocars. A laparascope that may or may not include a camera may be used through one of these ports for visualization of the anatomy and surgical instruments may be used simultaneously through other ports. Such devices and procedures permit a physician to position, manipulate, and view anatomy, surgical instruments and accessories inside the patient through a small access opening in the patient's body.

Still less invasive procedures include those that are performed through insertion of an endoscope through a natural body orifice to a treatment region. Examples of this approach include, but are not limited to, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user utilizing controls at the proximal end. Treatment or diagnosis may be completed intralumenally, such as polypectomy or gastroscopy. Alternatively, treatment or diagnosis of extra-luminal anatomy in the abdominal cavity may be completed translumenally, for example, through a gastrotomy, colonotomy or vaginotomy. Minimally invasive therapeutic procedures to treat or diagnose diseased tissue by introducing medical instruments translumenally to a tissue treatment region through a natural opening of the patient are known as Natural Orifice Translumenal Endoscopic Surgery (NOTES)™.

Some flexible endoscopes are relatively small (1 mm to 3 mm in diameter), and may have no integral accessory channel (also called biopsy channels or working channels). Other flexible endoscopes, including gastroscopes and colonoscopes, have integral working channels having a diameter of about 2.0 to 3.7 mm for the purpose of introducing and removing medical devices and other accessory devices to perform diagnosis or therapy within the patient. As a result, the accessory devices used by a physician can be limited in size by the diameter of the accessory channel of the scope used. Additionally, the physician may be limited to a single accessory device when using the standard endoscope having one working channel.

Over the years, a variety of different sheaths and overtubes for accommodating endoscopes and the like have been developed. Some sheath arrangements such as those disclosed in U.S. Pat. No. 5,325,845 to Adair are substantially steerable by means of control knobs supported on a housing assembly. Regardless of the type of surgery involved and the method in which the endoscope is inserted into the body, the surgeons and surgical specialists performing such procedures have generally developed skill sets and approaches that rely on anatomical alignment for both visualization and tissue manipulation purposes. However, due to various limitations of those prior overtube and sheath arrangements, the surgeon may often times be forced to view the surgical site in such a way that is unnatural and thereby difficult to follow and translate directional movement within the operating theater to corresponding directional movement at the surgical site. Moreover, such prior devices are not particularly well-equipped to accommodate and manipulate multiple surgical instruments and tools within the surgical site without having to actually move and reorient the overtube.

Consequently a significant need exists for an alternative to conventional overtubes and sheaths for use with endoscopes and other surgical tools and instruments that can be advantageously manipulated and oriented and which can accommodate a variety of different tools and instruments and facilitate movement and reorientation of such tools and instruments without having to reorient or move the outer sheath.

The foregoing discussion is intended only to illustrate some of the shortcomings present in the field at the time, and should not be taken as a disavowal of claim scope.

### SUMMARY

In one embodiment, a guide system for accommodating an endoscopic tool is disclosed. The guide system comprises a flexible inner sheath and a handle coupled to the inner sheath adjacent a proximal end of the inner sheath. The inner sheath includes a plurality of working channels. The working channels are bundled over a common portion of their respective lengths, and the working channels collectively define a substantially honeycombed cross-sectional area.

In another general embodiment, a surgical device comprising a flexible elongated inner sheath to be received through a body lumen is disclosed. The inner sheath includes a first length having at least one chamber inflatable to define one or more working channels.

### BRIEF DESCRIPTION OF THE FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 is a side view of one embodiment of a guide system;

FIG. 2 is a side view of one embodiment of an inner sheath;

FIG. 3 is a partial perspective view of one embodiment of a distal end portion of an inner sheath;

FIG. 4 is a partial perspective view of one embodiment of a distal end portion of an outer sheath;

FIG. 5 is a partial perspective view of the inner sheath of FIG. 3 inserted in the outer sheath of FIG. 4;

FIG. 6 is a partial perspective view of one embodiment of distal end portion an inner sheath;

FIG. 7 is an end view of one embodiment of an inner sheath inserted into an outer sheath;

FIG. 8 is a partial perspective view of one embodiment of a distal end portion of an inner sheath having locking detents formed thereon;

FIG. 9 is a partial perspective view of one embodiment of a distal end portion of an outer sheath having detent pockets formed therein;

FIG. 10 is a partial perspective view of the inner sheath embodiment of FIG. 8 inserted in the outer sheath embodiment of FIG. 9;

FIG. 11 is a diagrammatical view illustrating one embodiment of a guide system inserted through a patient's mouth and esophagus to perform a gastrotomy through the stomach wall;

FIG. 12 is a diagrammatical view of the guide system and patient's stomach of FIG. 11, with a conventional hole-forming device extending through a conventional endoscope supported in the guide system and forming a hole through the stomach wall;

FIG. 13 is a diagrammatical view of the guide system and patient's stomach depicted in FIGS. 11 and 12, with the inner sheath of the guide system protruding out of the outer sheath;

FIG. 14 is a diagrammatical view of the guide system and patient's stomach after a portion of the body cavity has been insufflated;

FIG. 15A is a perspective view of one embodiment of an inner sheath assembly;

FIG. 15B is a cross-sectional view of the working channels of the embodiment of FIG. 15A;

FIG. 16 is a cross-sectional view of one embodiment of working channels of the inner sheath;

FIG. 17 is a perspective view of one embodiment of a retainer of the inner sheath assembly;

FIG. 18 is a perspective view of one embodiment of an inner sheath assembly including a first actuator;

FIG. 19 is a perspective view of one embodiment of an inner sheath assembly including a second actuator;

FIG. 20A is a partial perspective view of one embodiment of an inner sheath assembly including a tip;

FIG. 20B is a proximal view of the tip of the embodiment of FIG. 20A;

FIG. 21A is a perspective view of one embodiment of an inner sheath assembly including a flexible core;

FIG. 21B is a cross-sectional view of the handle of the embodiment of FIG. 21A;

FIG. 21C is distal end view of the handle of the embodiment of FIG. 21A;

FIG. 22A is a partial perspective view of one embodiment of an inner sheath assembly including first and second working channel exit sites;

FIG. 22B is a partial perspective view of one embodiment of an inner sheath assembly including an articulation joint disposed between first and second working channel exit sites;

FIG. 23 is a partial perspective view of one embodiment of an inner sheath including a first length having at least one inflatable chamber;

FIG. 24 is a partial perspective view of one embodiment of an expandable partition;

FIG. 25 is a perspective view of one embodiment of an inner sheath including a non-inflatable second length;

FIG. 26A is a perspective view of one embodiment of an inner sheath including a guidewire channel;

FIG. 26B is a perspective view of one embodiment of the first length of the inner sheath in a deflated state and wrapped around the guidewire channel;

FIG. 27A is a perspective view of one embodiment of an inner sheath assembly;

FIG. 27B is an exploded view of the inner sheath assembly of FIG. 27A;

FIGS. 28A and 28B are front perspective and rear views, respectively, of the housing of the inner sheath assembly of FIG. 27A;

FIG. 29 is a side view of the housing and first actuator of the inner sheath assembly of FIG. 27A;

FIGS. 30A, 30B and 30C are views of a housing of an inner sheath assembly according to one embodiment;

FIGS. 31A, 31B and 31C illustrate engagement of the distal tip portion of an endoscopic instrument by a ramped guide surface of the housing of FIGS. 30A, 30B and 30C, according to various embodiments;

FIG. 32 is a bottom view of a distal portion of the inner sheath assembly of FIG. 27A;

FIG. 33 illustrates deployment of endoscopic instruments at a treatment site using the inner sheath assembly of FIG. 27A; and

FIG. 34 illustrates deployment of an endoscopic instrument using an embodiment of an inner sheath assembly including the housing of FIGS. 30A-30C.

### DETAILED DESCRIPTION

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of these embodiments is defined solely by the claims. The features illustrated or described in connection with one embodiment may be combined with the features of other embodiments. Such modifications and variations are intended to be included within the scope of the appended claims.

The various embodiments generally relate to various devices and overtube arrangements for use in connection with surgical instruments such as, for example, endoscopes for selectively positioning and manipulating endoscopic tools in a desired orientation within the body cavity. The term “endoscopic tools,” as used herein may comprise, for example, endoscopes, lights, insufflation devices, cleaning devices, suction devices, hole-forming devices, imaging devices, cameras, graspers, clip appliers, loops, Radio Frequency (RF) ablation devices, harmonic ablation devices, scissors, knives, suturing devices, etc. However, such term is not limited to those specific devices. As the present Description proceeds, those of ordinary skill in the art will appreciate that the unique and novel features of the various instruments and methods for use thereof may be effectively employed to perform surgical procedures by inserting such endoscopic tools through a natural body lumen (mouth, anus, vagina) or through a transcutaneous port (abdominal trocar, cardiothoracic port) to perform surgical procedures within a body cavity.

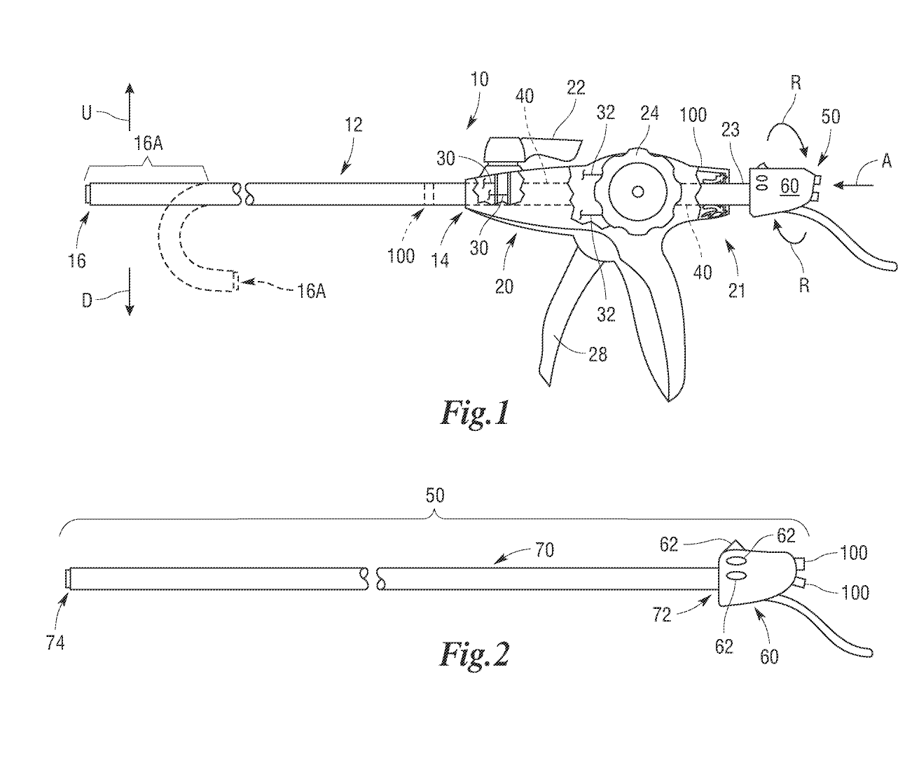

FIG. 1 illustrates an embodiment of a guide system 10 that comprises an outer sheath 12 having a proximal end 14 coupled to a handle assembly 20. It will be appreciated that the terms “proximal” and “distal” are used herein with reference to a clinician manipulating the handle assembly 20. The term “proximal” referring to the portion closest to the clinician and the term “distal” referring to the portion located away from the clinician. It will be further appreciated that for convenience and clarity, spatial terms such as “vertical”, “horizontal”, “up” and “down” may be used herein with respect to the drawings. However, surgical instruments are used in many orientations and positions, and these terms are not intended to be limiting and absolute.

As shown in FIG. 1, the elongated hollow outer sheath 12 may further have a distal end 16 that is substantially steerable by control knobs 22 and 24 operably supported on the handle assembly 20. For example, the control knob 22 may be operably coupled to a first pair of right/left cables 30 that extend through lumens (not shown) in the outer sheath 14 and are operably affixed to the distal end 16 of the outer sheath 14. Similarly, the control knob 24 may be operably affixed to up/down cables 32 that also extend through corresponding lumens (not shown) in the outer sheath 14 and are affixed to the distal end 16 thereof. Thus, rotation of the control knob 22 relative to the handle assembly 20 may cause the distal end 16 of outer sheath 12 to move in left and right directions (into and out of the page as depicted in FIG. 1) and rotation of the control knob 24 relative to the handle assembly 20 may cause the distal end 16 of the hollow outer sheath 12 to move up and down (arrows “U” and “D” in FIG. 1). A locking trigger 28 may be provided to selectively lock the distal end 16 in a desired position. Steerable sheaths and tube arrangements are known in the art and, therefore, their construction and use will not be discussed in great detail herein. For example, U.S. patent application Ser. No. 11/762,855 to James T. Spivey and Omar J. Vakharia, entitled CONTROL MECHANISM FOR FLEXIBLE ENDOSCOPE DEVICE AND METHOD OF USE, filed Jun. 14, 2007, which is commonly owned by the Assignee of the present application discloses such an arrangement and is herein incorporated by reference in its entirety. Another steerable sheath arrangement is disclosed in U.S. Pat. No. 5,325,845 to Aidar, issued Jul. 5, 1994, the entire disclosure of which is herein incorporated by reference.

In various embodiments, the hollow outer sheath 12 may be fabricated from, for example, plastic, TEFLON® or rubber inner/outer sheath material and a metallic, plastic, or composite coil pipe or extruded insertion tube which may provide some axial and rotational stiffness to allow for push/pull and rotation of the outer sheath. The articulation section 16A may be fabricated from, for example, a series of joined plastic, metallic, or composite links or from a plastic, metallic or composite tube with material removed in locations to allow articulation of the distal end 16 thereof in two axes and surrounded with material similar to the remainder of the outer sheath 12. The proximal end 14 of the hollow outer sheath 12 may be substantially coaxially aligned with a lumen 40 that extends through the handle assembly 20 such that an inner sheath assembly 50 may be inserted through an opening 23 in the proximal end 21 of the handle assembly 20, through lumen 40 and into the hollow outer sheath 12 as illustrated in FIG. 1. In various embodiments, the inner sheath assembly 50 comprises a control head 60 that has a substantially flexible inner sheath 70 attached thereto. The inner sheath may be fabricated from, for example, plastic, TEFLON® or rubber inner/outer sheath material and a metallic, plastic, or composite coil pipe or extruded insertion tube and have a proximal end 72 that is attached to the control head 60. The inner sheath 70 may further have a distal end 74 and be configured relative to the hollow outer sheath 12 such that the inner sheath 70 may be selectively rotatable and axially movable within the outer sheath 12 as represented by arrows “A” and “R” in FIGS. 1 and 5. The inner sheath 70 may also be sized and configured relative to the outer sheath 12, for example, such that the distal end 74 of the inner sheath 70 may protrude out beyond the distal end 16 of the outer sheath 12 as shown in FIG. 5. Those of ordinary skill in the art will appreciate that such arrangement not only enables the distal end 74 of the inner sheath 70 to be advantageously positioned, but the distal end 74 can also be used to move and manipulate tissue as needed.

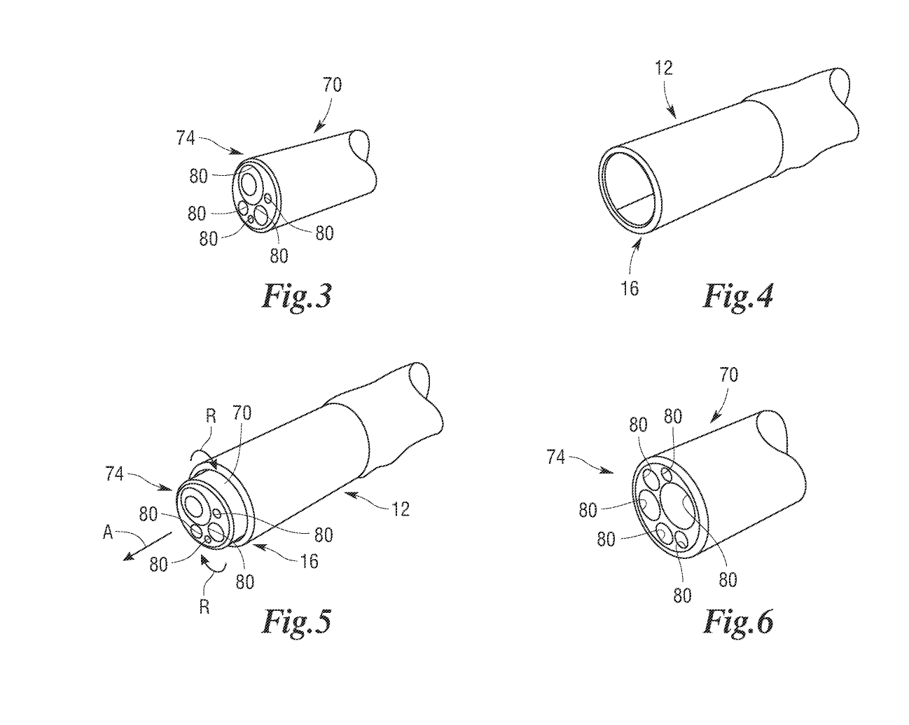

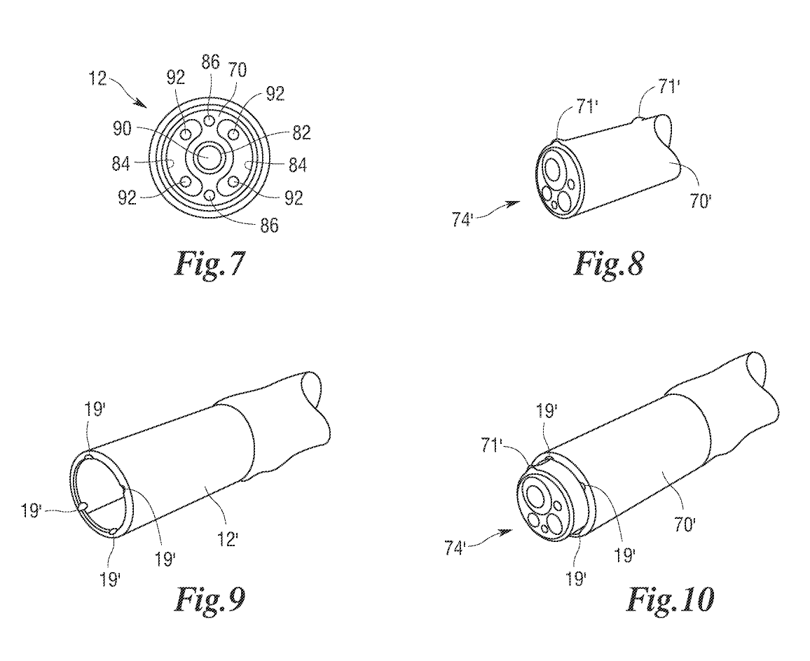

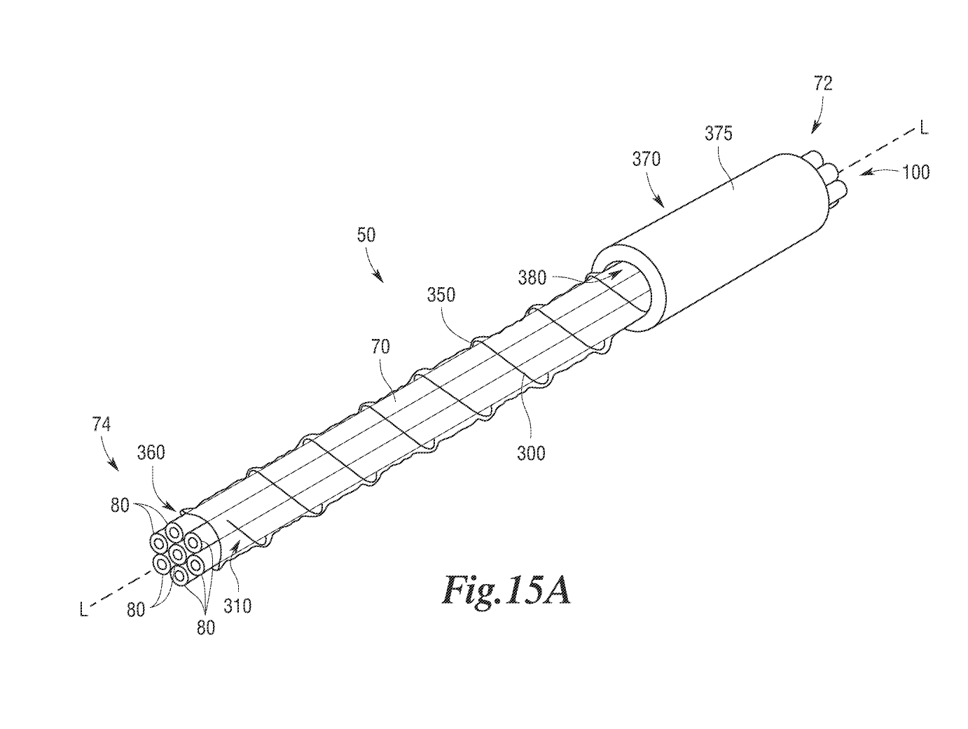

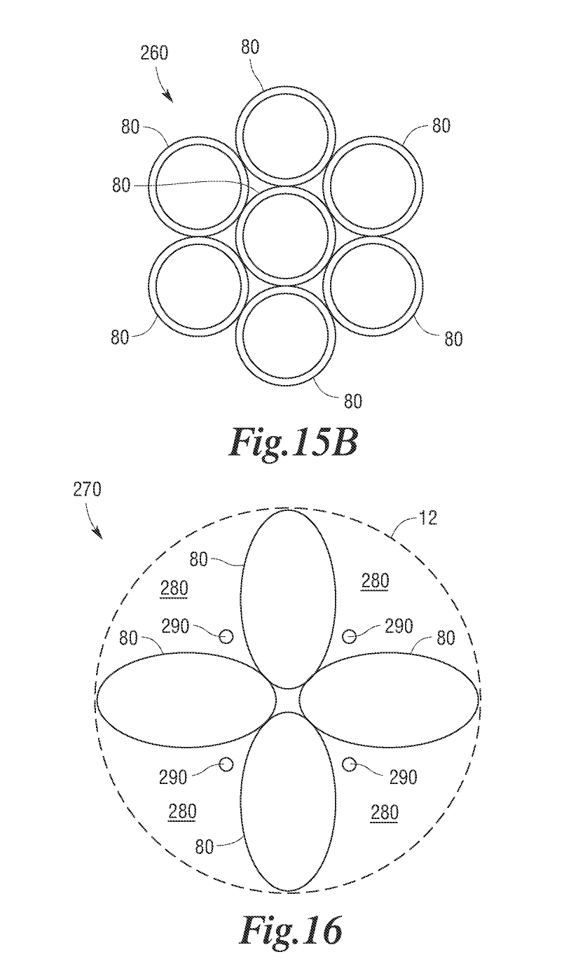

As shown in FIGS. 3 and 5, the inner sheath 70 may have at least one, and preferably a plurality of, working channels 80 formed therein. The working channels 80 may vary in number, size, and shape. For example, in the embodiment depicted in FIG. 3, the inner sheath 70 has five working channels 80 therein that vary in size, but all have a substantially circular cross-section. In the embodiment depicted in FIG. 6, the inner sheath 70 has six working channels 80 of various sizes. In the embodiment depicted in FIG. 7, the inner sheath 70 has a somewhat “honeycombed” cross-sectional configuration. In that embodiment, a central lumen or working channel 82 is provided though the inner sheath 70. Such central lumen 82 may, for example, operably support a camera 90 therein. Oriented around the central lumen 82 are two “oblong” working channels 84 that may, for example, each support a plurality of endoscopic tools 92 (hole-forming devices, light bundles, imaging devices, cameras, graspers, clip appliers, loops, Radio Frequency (RF) ablation devices, harmonic ablation devices, scissors, knives, suturing devices). This embodiment also includes smaller working channels 86 that may facilitate the introduction of an insufflation medium (for example, air or carbon dioxide, fluid, such as, for example, water, saline solution, sterile solution, alcohol, betadine, staining inks, staining dyes into the body area adjacent the target tissue. Other embodiments incorporating honeycombed cross-sectional configurations and other features are discussed below in connection with FIGS. 15A-26B.

In some applications, it may be advantageous to essentially lock the inner sheath in a predetermined position relative to the outer sheath. For example, as can be seen in FIGS. 8-10, the inner sheath 70′ may have one or more than one detents 71′ formed thereon that may be received in corresponding pockets 19′ provided in the distal end 16′ of the outer sheath 12′. Thus, the inner sheath 70′ may be rotated to a predetermined position defined by the corresponding pockets 19′ and retained in that position relative to outer sheath 12′ by bringing the corresponding detent 71′ into locking engagement with the corresponding pocket 19′. Those of ordinary skill in the art will understand that such locking arrangement may be provided in a variety of different forms without departing from the spirit and scope of the present invention. For example, in an alternative embodiment, the detents may be provided in the outer sheath and the pockets may be provided in the inner sheath. In other embodiments, the detents may extend substantially the entire length of the sheath and the pockets may each comprise an axial groove that also extends substantially the entire length of the sheaths. Different numbers, shapes and sizes of detents and/or pockets may also be employed.

In various embodiments, one or more seals 100 may be employed to achieve a substantially airtight/fluidtight seal around the inner sheath 70. For example, a seal 100 may be provided in the handle assembly 20 to achieve an airtight/fluidtight seal between the inner sheath 70 and the lumen 40 in the handle assembly 20. In addition to, or in the alternative, a seal 100 may be provided in the outer sheath 12 to achieve a substantially fluidtight or airtight seal between the inner sheath 70 and the outer sheath 12. A variety of existing seal arrangements may be employed. For example, U.S. Pat. No. 5,401,248, entitled SEAL FOR TROCAR ASSEMBLY, issued Mar. 28, 1995 to Bencini and U.S. Pat. No. 7,163,525, entitled DUCKBILL SEAL PROTECTOR, issued Jan. 16, 2007, the disclosures of which are each incorporated by reference herein in their respective entireties, disclose seals that may be employed to establish a substantially airtight/fluidtight seal between the inner sheath 70 and outer sheath 12. The working channels 80 in the inner sheath 70 may also each be fitted with a similar seal 100 such that when the working channel 80 is not being used, the working channel 80 is sealed off and when an endoscopic tool is inserted into the working channel 80, a substantially airtight/fluidtight seal is achieved between the endoscopic tool and the working channel 80. In various embodiments, for example, the seals 100 may be mounted on the control head 60 as shown in FIG. 2.

The working channels 80, 84, 86 may be used to apply suction, pressurized air, fluid to an area within the body. The control head 60 of the inner sheath assembly 50 may be provided with a series of control buttons 62, or the like, that serve to control various endoscopic tools or instruments inserted therethrough. For example, such control buttons 62 may be used to control the application of suction, insufflation mediums, cleaning mediums. Such buttons may also consist of buttons for controlling lights, zooming of the camera.

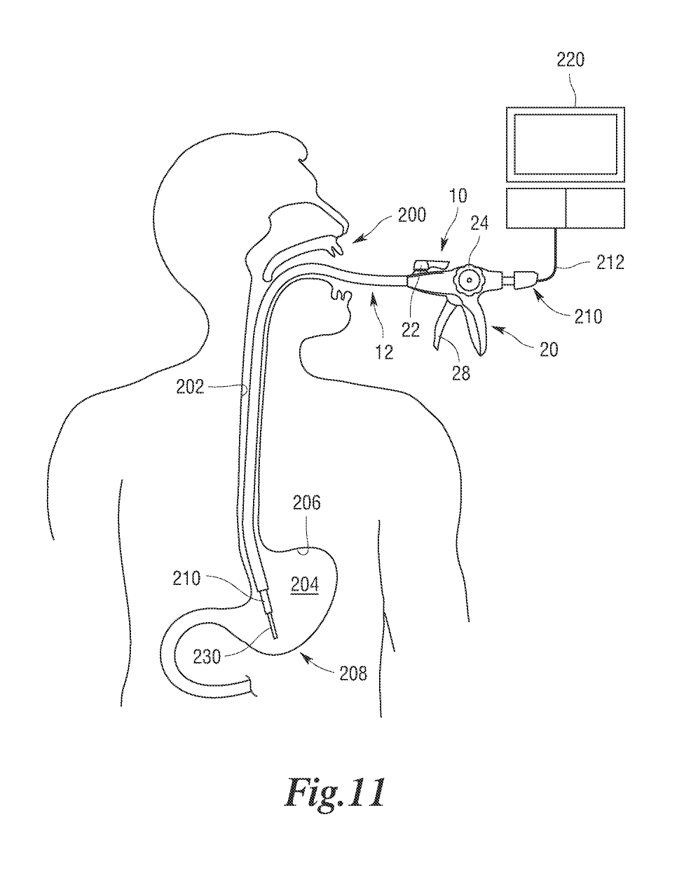

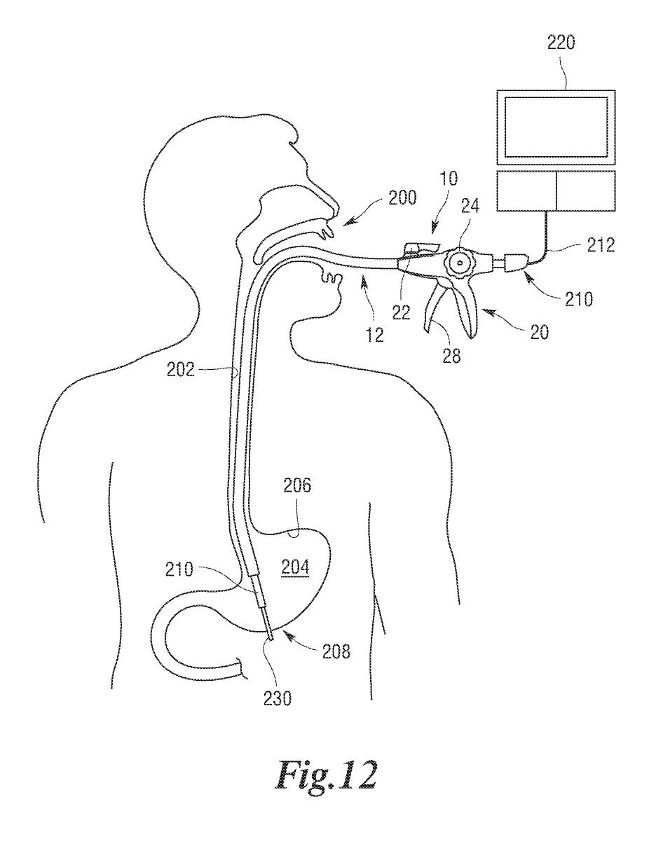

FIGS. 11-14 illustrate various embodiments of methods of using the guide system 10 of the present invention. As shown in FIG. 11, the outer sheath 12 can be inserted through a natural orifice to form an opening through the stomach wall 206. In the example depicted in FIGS. 11-14, the outer sheath 12 is inserted through the mouth 200 and esophagus 202 into the stomach 204 to form an opening through the stomach wall 206. During this procedure, the clinician may manipulate the distal end 16 of the outer sheath 12 by means of the control knobs 22 and 24 as needed. Once the outer sheath 12 has been oriented in a desired position, the clinician may lock the outer sheath 12 in that position by engaging the locking trigger 28 on the handle assembly 20. The clinician may insert a conventional active or passive endoscope 210 that has a camera and a working channel therein through the outer sheath 12 as shown in FIG. 11 to locate the portion of the stomach wall 206 (or target tissue 208) through which the hole is to be made. The endoscope 210 may be attached to a viewing screen 220 in the operating suite by an umbilical cord 212. Once the target tissue 208 has been located and the endoscope 210 properly positioned, the clinician may insert a conventional hole-forming instrument 230 through the working channel in the endoscope 210 to form a hole 209 through the target tissue 208. See FIG. 12. After the hole 209 has been formed through the target tissue 208 and the outer sheath has been inserted through the hole, the endoscope 210 and hole-forming instrument 230 may be removed from the outer sheath 12.

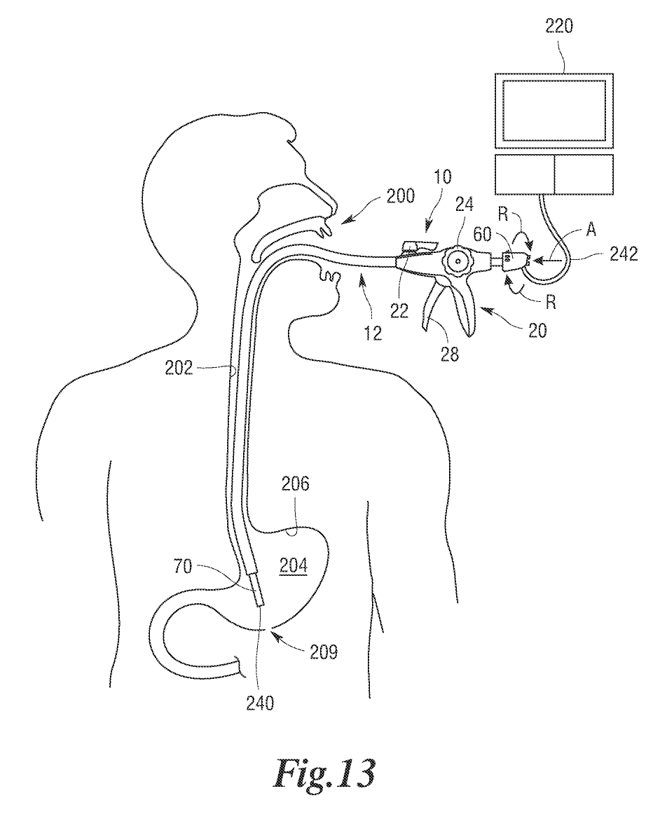

The clinician may then insert the inner sheath 70 in through the outer sheath 12 as shown in FIG. 13. A smaller camera 240 may be supported in one of the working channels in the inner sheath 70 and be coupled to the screen 220 by an umbilical cord 242. The distal end 74 of the inner sheath 70 may be axially advanced out of the distal end 16 of the outer sheath 12 as shown in FIG. 13 and rotated as necessary until the clinician attains a desired or familiar picture orientation on the screen 220. During this process, the clinician may use the distal end 74 of the inner sheath 70 to manipulate/position tissue as needed. Once in a desired position, the clinician may lock the inner sheath 70 relative to the outer sheath 12 by bringing the detent(s) into retaining engagement with corresponding pocket(s). Those of ordinary skill in the art will appreciate that the smaller camera 240 may also be advanced out through the distal end 74 of the inner sheath 70 as necessary. Alternatively, the clinician may initially use the inner sheath 70 with the smaller camera 240 to obtain access through the stomach wall 206, thus obviating the need from a standard endoscope for access.

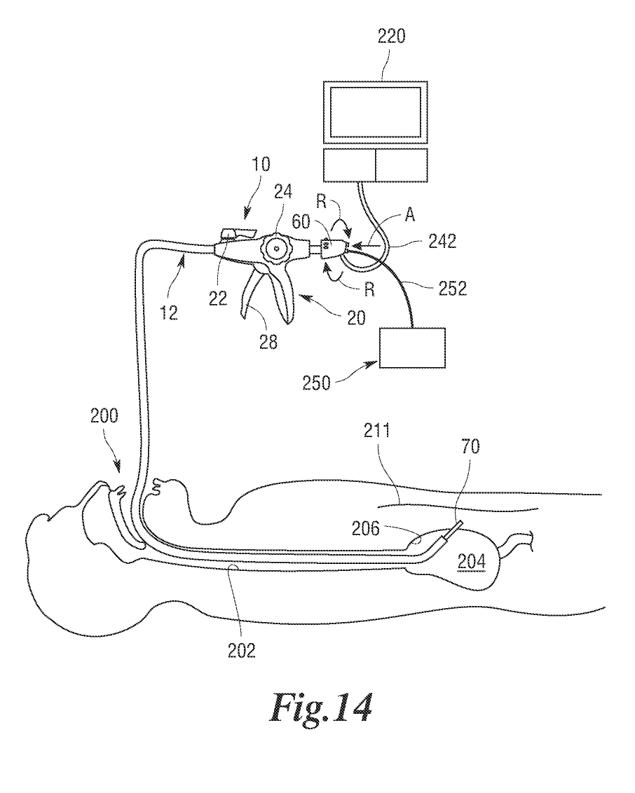

The medical procedure may further require the body cavity 211 to be insufflated. To accomplish this procedure, an insufflation medium such as, for example, air or carbon dioxide may be introduced into the body cavity portion 211 through a working channel in the inner sheath 70. Such insufflation medium may be supplied through a supply line 252 that has been inserted into a working channel in the inner sheath 70 and is coupled to a source of insufflation medium 250. The insufflation medium is supplied through the supply line 252 extending through the working channel and, once the desired pressure is attained, a standard operating room insufflation controller can be used to maintain the desired pressure via the supply line 252. See FIG. 14. The clinician may then insert other endoscopic tools through the working channels in the inner sheath 70 to perform various procedures. One of ordinary skill in the art will understand that the various seal arrangements employed in the guide system 10 facilitate maintenance of the insufflation within cavity portion 211 while additional tool(s)/instrument(s) are inserted and manipulated therein. It will be further appreciated that the inner sheath 70 may also be advantageously repositioned, axially moved, rotated during the operation as need to provide the clinician with the desired tool/instrument positioning and support as well as the desired video display orientation on the screen 220. This feature may be particularly useful to the clinician who is most familiar with a particular tissue orientation, for example, the tissue orientation that is often depicted in medical journals, books and reference materials or commonly addressed through open or laparoscopic surgical means. Alternatively, insufflation may be achieved and maintained through space between the inner sheath 70 and the outer sheath 12. For example, an insufflation connection may be made at the handle assembly 20 connected to the outer sheath 12, and pressure may be maintained by a suitable seal located between the handle assembly 20 and the inner sheath 70.

FIG. 15A illustrates one embodiment of an inner sheath assembly 50. As shown, the inner sheath assembly 50 may comprise an inner sheath 70 including a plurality of working channels 80 bundled over a common portion of their respective lengths to define a honeycombed cross-sectional area, i.e., a cross-sectional area comprising a plurality of cells closely packed such that a portion of each cell wall abuts a wall portion of at least one neighboring cell. FIG. 15B illustrates a honeycombed cross-sectional area 260 of the plurality of working channels 80 of FIG. 15A taken at an angle transverse to a longitudinal axis L defined by the inner sheath assembly 50. It will be appreciated that the area and shape of the cells of the honeycombed cross-sectional area 260 is determined by the cross-sectional area and cross-sectional shape of the working channels 80, as well as the orientation of the working channels 80 relative to the longitudinal axis L of the inner sheath assembly 50. In certain embodiments and as shown in FIG. 15A, for example, the working channels 80 may comprise generally cylindrical tubes of uniform diameter and be generally aligned with the longitudinal axis L of the inner sheath assembly 50. Accordingly, the cells of the honeycombed cross-sectional area 260 of FIG. 15B are circular and of the same diameter. It will be appreciated, however, that the working channels 80 may generally comprise any cross-sectional area or combination of areas, any cross-sectional shape (e.g., oval, square) or combination of shapes, and that the working channels 80 may be oriented in parallel or non-parallel orientations (e.g., twisted, interwoven) relative to the longitudinal axis L of the inner sheath assembly 50. It will therefore be appreciated that the cells of the honeycombed cross-sectional area 260 may generally comprise any shape or combination of shapes and have identical or varying areas. It further will be appreciated that while the inner sheath 70 of FIG. 15A is depicted as comprising seven working channels 80, the number of working channels 80, and thus the number of cells of the honeycombed cross-sectional area 260, may generally be two or more.

In certain embodiments, and as shown in FIG. 15A, the bundled length of the inner sheath 70 may substantially include the distal ends of the working channels 80 such that the distal ends are collectively positionable. In other embodiments, the distal end of at least one working channel 80 may be unbundled so that the distal end may be articulated and positioned independently of other working channels 80.

In certain embodiments and as shown in FIG. 15B, the number, cross-sectional shape(s) and cross-sectional areas of the working channels 80 may be such that the inner sheath 70 defines a circular, or substantially circular, honeycombed cross-sectional area 260. In other embodiments, the number, cross-sectional shape(s) and cross-sectional areas of the working channels 80 may be varied such that the inner sheath 70 defines a non-circular honey-combed cross-sectional area. For example, with reference to FIG. 16, the inner sheath 70 may comprise four working channels 80 having generally ovular cross-sectional shapes of equal area that collectively define a clover-leaf shaped honeycombed cross-sectional area 270. In such embodiments, resulting void(s) 280 between the inner sheath 70 and the outer sheath 12 resulting from the non-circular shape of the inner sheath 70 may be used, for example, to introduce carbon dioxide or other gas or substance into a body cavity for purposes of insufflation. Additionally or alternatively, the void(s) 280 may be used to accommodate other devices or materials. In certain embodiments and as shown in FIG. 16, for example, one or more light fibers 290 for supplying light to the distal end 74 of the inner sheath 70 may be contained within each void 280.

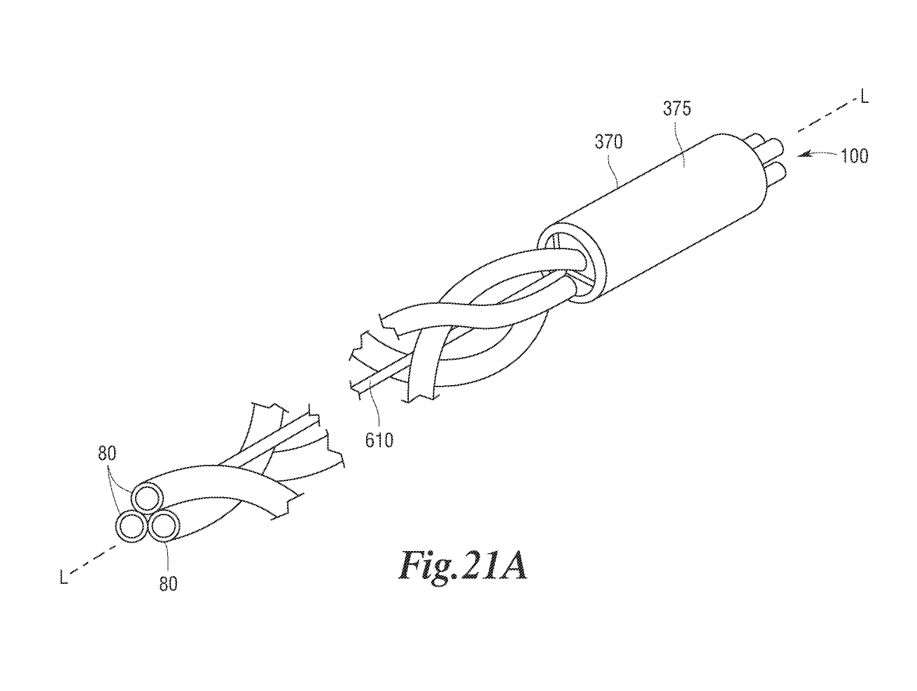

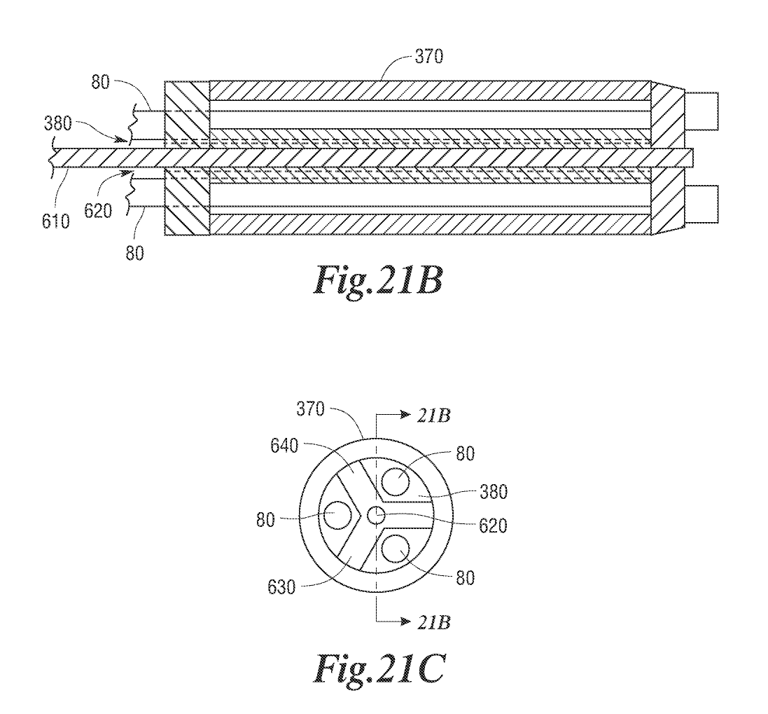

According to various embodiments, the inner sheath assembly 50 may comprise at least one retainer disposed over a length of the inner sheath 70 to retain the plurality of working channels 80 in a substantially fixed orientation relative to each other. In certain embodiments, and as shown in FIG. 15A, for example, the inner sheath assembly 50 may comprise a retainer in the form of a flexible coil 300 defining a longitudinal bore 310 to receive the plurality of working channels 80. The coil 300 may be, for example, an open coil spring (as shown), or a closed coil spring, and may be constructed from a suitably flexible metal or plastic and have a number of turns per unit length to suitably retain the plurality of working channels 80 in a honeycombed configuration. Torsional characteristics of the coil 300 may be selected to define a desired torsional response of the inner sheath assembly 50. Additionally, or alternatively, torsional response may be defined, for example, by collectively twisting the working channels 80 in a particular direction about the longitudinal axis L of the inner sheath assembly 50. In certain embodiments, the working channels 80 may be twisted in a direction opposite a twist of the coil 300. For example, if the coil 300 comprises a right-hand twist, the working channels 80 may be twisted with a left-hand twist. In this way, the torsional response of the inner sheath assembly 50 may be balanced to an extent. Additionally, twisting of the working channels 80 may be used to generally enhance the flexibility of the inner sheath. In certain embodiments, the coil 300 may comprise features (e.g., loops formed on one or more of the turns within the bore 310, one or more spoked inserts contained within the bore 310) for accommodating and retaining the flexible core 610 (FIG. 21A) of the inner sheath assembly 50 within the coil's bore 310.

Although the coil 300 of FIG. 15A comprises a generally circular cross-sectional area about its longitudinal axis to accommodate the similarly-shaped honeycombed cross-sectional area 260 of the inner sheath 70, it will be appreciated that the coil 300 may generally comprise any cross-sectional shape. For example, in embodiments in which the honeycombed cross-sectional area is non-circular (e.g., the clover-leaf shaped cross-sectional area 270 of FIG. 16), the cross-section of the coil 300 may be correspondingly shaped. In such embodiments, the coil 300 may be configured to aid the retention of devices or materials (e.g., light fibers) contained in the void(s) 280 between the inner sheath 70 and the outer sheath 12 (FIG. 16).

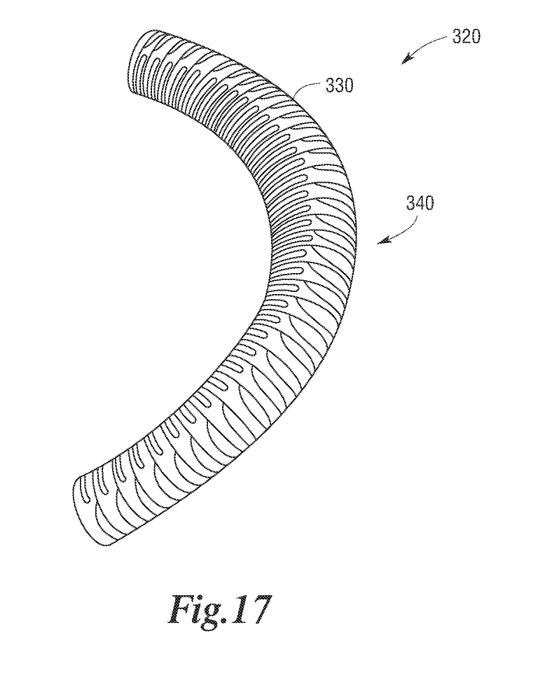

With reference to FIG. 17, as an alternative to the flexible coil 300, the inner sheath assembly 50 may comprise a retainer in the form of a flexible tube 320 including an elongate hollow body 330 defining a central opening suitable for receiving the plurality of working channels 80 therethrough. A series of slits 340 may be formed into the body 330 to define a plurality of articulatably interconnected elements to make the tube 320 flexible while still providing sufficient column strength and torque transmission characteristics. In certain embodiments, the series of slits 340 may be formed to limit the degree of articulation of the body 330 or a portion of the body 330 to range of pre-determined angles. The flexible tube 320 may be formed of a variety of materials including metallic materials, steel, brass, polycarbonate, polyetheretherketone (PEEK), urethane, or polyvinylchloride (PVC). In one embodiment, the flexible tube 320 may be constructed of full-hardened steel that tends to spring back more readily than softened annealed metal. In one embodiment, the series of slits 340 may be formed with a laser cutter. In other embodiments, the series of slits 340 may be formed with a machine bit or other suitable means for forming a substantially narrow cut, opening, or aperture, for example. In one embodiment, the series of slits 340 may be cut into the body 330 in a predetermined pattern without removing sections or portions of the material other than the kerf. In another embodiment, the series of slits 340 may be formed by removing sections or portions of the material along its length. In yet another embodiment, the series of slits 340 may be formed by creating a mold of a desired form and shape and then molding the tube 320 using conventional plastic molding techniques. It will be appreciated that any combination of these techniques may be employed to form the series of slits 340 in a predetermined pattern defining a plurality of articulatable elements that render the tube 320 flexible yet sufficiently rigid to provide suitable column strength and torque transmission characteristics. The construction of flexible tubes according to these and other embodiments is disclosed in U.S. application Ser. No. 12/172,782 to Spivey et al. filed Jul. 14, 2008 and entitled ENDOSCOPIC TRANSLUMENAL ARTICULATABLE STEERABLE OVERTUBE, the disclosure of which is incorporated herein by reference. In certain embodiments, the flexible tube 320 may comprise features (e.g., loops formed within one or more of the articulatable elements, one or more spoked inserts contained within the flexible tube 320) for accommodating and retaining the core 610 (FIG. 21A) of the inner sheath assembly 50 within the tube 320.

In certain embodiments, in addition to the flexible coil 300 or flexible tube 320, the inner sheath assembly 50 may comprise a retainer in the form of a flexible sleeve 350 defining a longitudinal bore 360 to receive the plurality of working channels 80. With reference to FIG. 15A, for example, the sleeve 350 may be conformably disposed over the coil 300 to provide a fluid-tight and gas-tight prophylactic barrier that compresses the underlying structures to a degree but does not significantly lessen their flexibility. The sleeve 350 may generally comprise any flexible material that is conformable to the coil 300 or the tube 320 and suitably impermeable to fluids and gases. For example, in certain embodiments, the sleeve 350 may comprise polyolefin heat shrink tubing, dual-wall heat shrink tubing having an inner melt layer and an outer heat shrink layer, an expandable PTFE sleeve, or an extruded rubber boot.

According to various embodiments, the inner sheath assembly 50 may comprise a handle 370 coupled to a proximal end 72 of the inner sheath 70. The handle 370 may comprise a gripping surface 375 for allowing a user to apply rotational and axial forces to the inner sheath 70. In certain embodiments, and as shown in FIG. 15A, the handle 370 may be generally cylindrical in shape and define a first bore 380 through which the proximal end 72 of the inner sheath 70 may extend. At least a portion of the one or more retainers (if present) may extend into a distal end of the first bore 380 and attach to its inner diameter, thus providing mechanical coupling between the handle 370 and the working channels 80 retained by the retainer(s). In certain embodiments, the one or more retainers may terminate within the first bore 380, and proximal ends of the working channels 80 may be relatively flush with the proximal end of the handle 370. Thus, addition to providing a gripping surface 375, the handle 370 may maintain proximal ends of the working channels 80 relatively straight to ensure straight passage of endoscopic tools into the working channels 80. It will be appreciated, however, that one or more working channels 80 may protrude beyond a proximal end of the handle 370. In certain embodiments, the gripping surface 375 of the handle 370 may be constructed from a dense plastic and may comprise contours for enhanced gripablity. Additionally or alternatively, the handle 370 may comprise a relatively soft material (e.g., urethane) that conforms to a user's hand and is slip resistant.

As discussed above in connection with the embodiment of FIG. 2, each of the plurality of working channels 80 may comprise a seal 100 such that when the working channel 80 is not being used, the working channel 80 is sealed off, and when an endoscopic tool is inserted into the working channel 80, a substantially airtight/fluidtight seal is achieved between the endoscopic tool and the working channel 80. In various embodiments, for example, the seals 100 may be mounted to the proximal end of the handle 370, as shown in FIG. 15A.

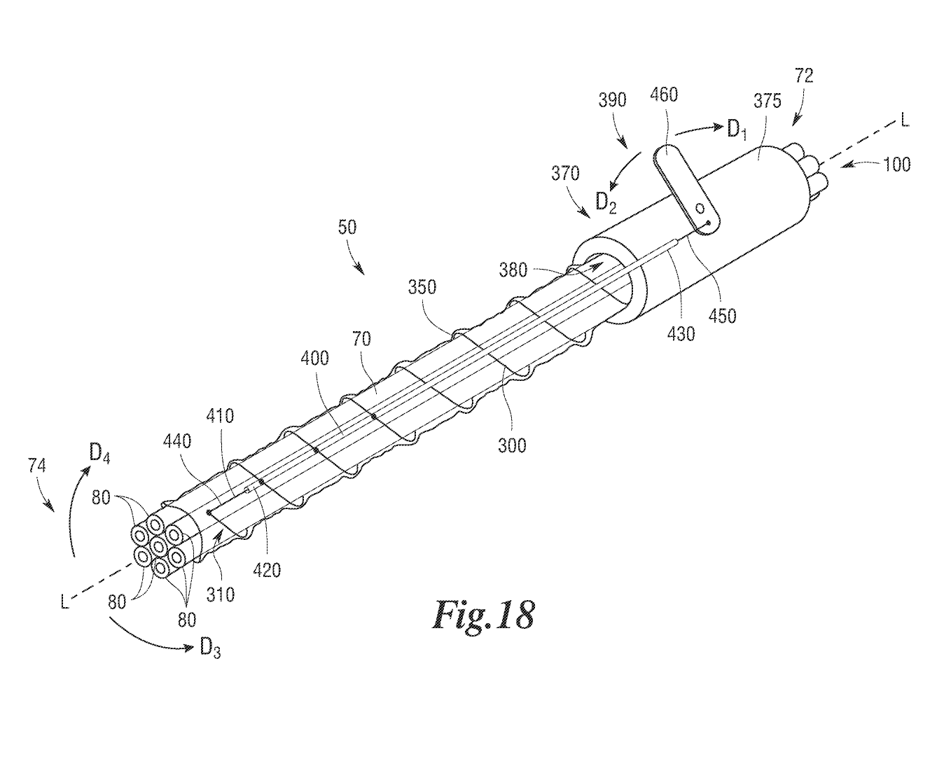

According to various embodiments, the inner sheath assembly 50 may comprise at least one first actuator 390 to position a distal end 74 of the inner sheath 70. For example, in embodiments of the inner sheath assembly 50 comprising a retainer in the form of a flexible coil 300, each first actuator 390 may comprise a flexible guide 400 extending over a length of the inner sheath 70, and a control member 410 slidably disposed within the guide 400. FIG. 18 illustrates an example of one such embodiment. As shown, the guide 400 may comprise an elongated tube constructed from, for example, a suitable plastic or a plastic-coated close-wound wire helix, and the control member 410 may comprise a single wire or, alternatively, a cable comprising a plurality of stranded wires and/or other stranded material. The guide 400 may comprise a distal end 420 attached to coil 300 at one or more locations adjacent its distal end, and a proximal end 430 adjacent the handle 370. The control member 410 may comprise a distal end 440 extending from the distal end 420 of the guide 400 and attached to the distal end of the coil 300, and a proximal end 450 extending from the proximal end 430 of the guide 400. Each first actuator 390 may further comprise a control device 460 attached to the proximal end 450 of the control member 410 for slidably translating the control member 410 though the guide 400 to position the distal end 74 of the inner sheath 70. The control device 460 may comprise a suitable mechanical or electromechanical actuator (e.g., a lever actuator, a knob actuator, a trigger actuator, a bar clamp actuator, a syringe grip actuator, a solenoid actuator, a motor actuator) for causing the control member 410 to translate within the guide 400. In FIG. 18, the control device 460 of the first actuator 390 is depicted as a lever actuator movable in directions D₁ and D₂ to move the distal end 74 of the inner sheath 70 in directions D₃ and D₄, respectively. In certain embodiments and as shown, the control device 390 may be configured for attachment to the handle 370. It will be appreciated that the inner sheath assembly 50 may comprise two or more first actuators 390 that cooperate to enhance the positionability of the distal end 74 of the inner sheath 70.

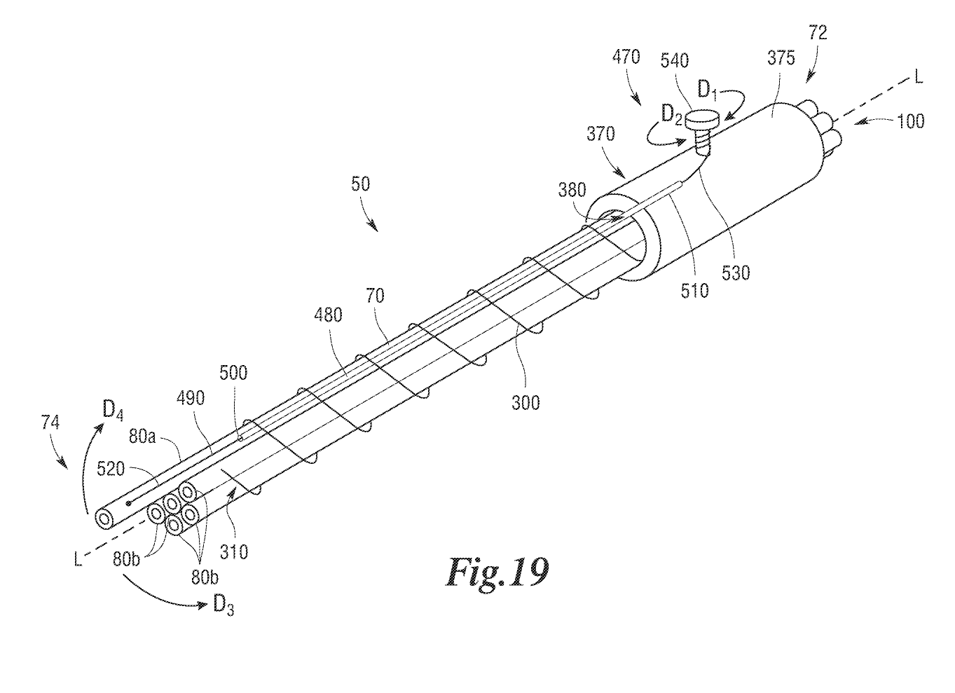

According to various embodiments, the inner sheath assembly 50 may comprise at least one second actuator 470 to position a distal end of at least one first working channel 80a relative to a distal end of one or more second working channels 80b. FIG. 19 illustrates an example of one such embodiment. Each second actuator 470 may comprise components similar to the first actuator 390 described above. For example, each second actuator 470 may comprise a flexible guide 480 and a control member 490 slidably disposed in the flexible guide 480, with the construction of the flexible guide 480 and control member 490 identical or similar to those of the first actuator 390. The flexible guide 480 of each second actuator 470 may extend over a length of the first working channel 80a and comprise a distal end 500 adjacent a distal end of the first working channel 80a, and a proximal end 510 adjacent the handle 370. The control member 490 may comprise a distal end 520 extending from the distal end 500 of the flexible guide 480 and attached to the distal end of the first working channel 80a, and a proximal end 530 extending from the proximal end 510 of the guide 480. Each second actuator 470 may further comprise a control device 540 attached to the proximal end 530 of the control member 490 for slidably translating the control member 490 though the guide 480 to position the distal end of first working channel 80a. As with the first actuator 390, the control device 540 may comprise any suitable mechanical or electromechanical actuator for causing the control member 490 to translate within the guide 480. In FIG. 19, the control device 540 of the second actuator 470 is depicted as a knob actuator rotatable to gather or release the control member 490 based on a direction of rotation. For example, rotational directions D₁ and D₂ result in movement of the distal end 74 of the first working channel 80a in directions D₃ and D₄, respectively. In certain embodiments and as shown, the control device 540 may be configured for attachment to the handle 370. It will be appreciated that the inner sheath assembly 50 may comprise two or more second actuators 540 that cooperate to enhance the positionability of a distal end of a first working channel 80a.

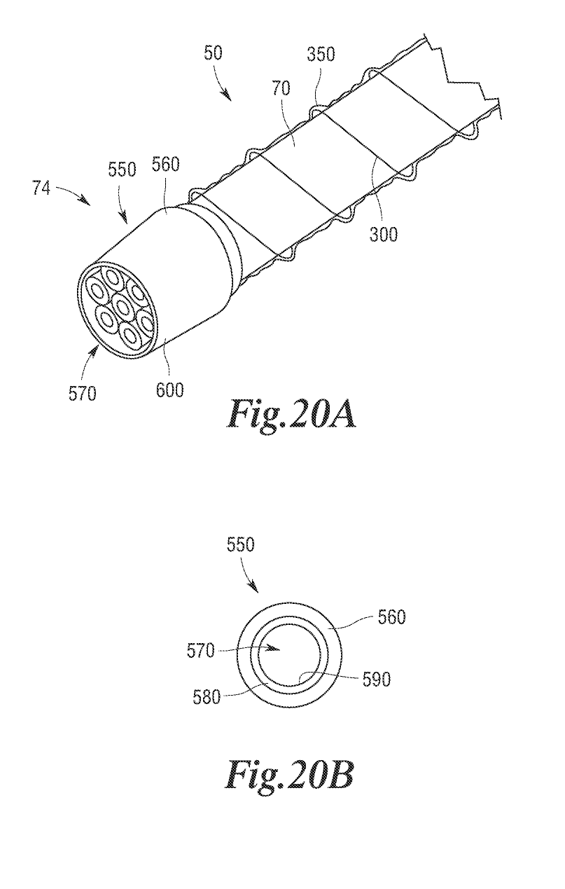

According to various embodiments and as shown in FIG. 20A, the inner sheath assembly 50 may comprise a tip 550 disposed over the distal end 74 of the inner sheath 70. The tip 550 may be, for example, an atraumatic tip shaped to facilitate passage of the inner sheath 70 through the outer sheath 12 and to reduce the risk of injury when the distal end 74 of the inner sheath 70 is introduced to a body lumen or treatment site. In certain embodiments, the tip 550 may comprise a relatively rigid external body 560 defining a bore 570 therethrough. A relatively soft insert 580 (FIG. 20B) may extend at least partially through the bore 570 from its proximal end and comprise a gripping surface 590. Insert 580 and gripping surface 590 may be provided for removably affixing the tip 550 to the inner sheath 70, such that tip 550 may be pushed onto the distal end 74 of the inner sheath 70 and removed therefrom without the need for special tools or assembly techniques. One or more of the insert 580 and gripping surface 590 may comprise a sticky or tacky material such as silicone or neoprene, or a suitable adhesive, to retain the tip 550 in place on the distal end 74 of the inner sheath 70. Alternatively, the tip 550 may be removably affixed to the distal end 74 of the inner sheath 70 using a snap fit, an interference fit, or any other suitable non-permanent attachment means. In certain embodiments, the tip 550 material may be sufficiently elastic such that the proximal opening of the bore 570 may be stretched or otherwise expanded to accommodate the distal end 74 of the inner sheath 70 and be retained thereon by frictional force, thus possibly eliminating the need for the insert 580.

Referring again to FIG. 20A, the external body 560 of the tip 550 may be made from a biocompatible plastic, such as, for example, nylon 6/6, polycarbonate, or polyvinylchloride (PVC), and may comprise a distal tip portion 600. The distal tip portion 600 of the tip 550 may have a variety of configurations depending on the intended use. In certain embodiments, at least a portion of the distal tip portion 600 may be constructed using a material that is suitably transparent or clear to allow an image gathering unit positioned within a working channel 80 to view and gather images through the distal tip portion 600. In certain embodiments, the distal tip portion 600 may be configured to enlarge an opening in tissue as it is advanced therethrough and/or to effect localized retraction, for example, by pushing the distal tip portion 600 onto an area of a treatment site. In certain embodiments, the distal tip portion 600 may be made of a soft, compressible material. In certain embodiments, the distal-most edge of the distal tip portion 600 may comprise an oblique or non-oblique contour.

As shown in FIG. 20A, the distal ends of the working channels 80 may be operatively positioned within the bore 570 adjacent the distal tip portion 600. Additionally or alternatively, one or more of the working channels 80 may be extendible though the distal opening of the bore 570.

According to various embodiments and as shown in FIG. 21A, the inner sheath assembly 50 may comprise at least one flexible core 610 attached to the handle 370 and distally extending therefrom. In certain embodiments and as shown, a portion of each working channel 80 may be tightly wrapped around the core 610 so that rotational force and/or translational force (e.g., force along the longitudinal axis L) applied to the handle 370 is at least partially transferred to the plurality of working channels 80 via the core 610. Although the embodiment of FIG. 21A does not include one or more retainers disposed over a length of the inner sheath 70, it will be appreciated that embodiments of the inner sheath assembly 50 comprising the core 610 may also include a retainer in the form of, for example, a flexible coil 300 or a flexible tube 320 as described above, through which the plurality of working channels 80 and the core 610 may be received. As discussed above, in certain embodiments the flexible coil 300 and the flexible tube 320 may comprise features (e.g., loops, spoked inserts) for accommodating and retaining the core 610. Optionally, a retainer in the form of, for example, a flexible sleeve 350 conformably disposed over the coil 300 or tube 320 as described above, may also be included. Alternatively, embodiments of the inner sheath assembly 50 comprising the core 610 may comprise a single retainer in the form of the flexible sleeve 350 conformably disposed directly over the plurality of working channels 80.

In certain embodiments, the core 610 may comprise a solid shaft fabricated from a suitable metal (e.g., carbon steel, stainless steel) or other suitable material. In one embodiment, for example, the core 610 may be implemented as a flexible solid shaft available from S.S. White Technologies Inc., Piscataway, N.J. that is fabricated from medium carbon spring steel with an outside diameter of about 0.071 inches.

In other embodiments, the core 610 may comprise a solid cable fabricated from a plurality of metal wire strands (e.g., stainless steel, platinum), or strands of another suitable material. In one embodiment, for example, the core 610 may be implemented using torque wirerope available from Asahi Intecc Co. Ltd., Aichi, Japan.

In yet other embodiments, the core 610 may comprise a hollow tube fabricated from a suitable metal (e.g., stainless steel, platinum) or other suitable material. In certain embodiments, for example, the core 610 may be implemented using round-wire coil, flat-wire coil, or a wire-stranded hollow tube, each available from Asahi Intecc Co. Ltd., Aichi, Japan. Alternatively, the core 610 may comprise a flexible tube having features similar or identical to those described above in connection with the flexible tube 320 of FIG. 17.

FIG. 21B is a cross-sectional view of the handle 370 configured for attachment to the core 610 according to one embodiment. The handle 370 may define a second bore 620 at least partially extending through the handle 370 from its distal end. The proximal end of the core 610 may be received into the distal end of the second bore 620 and retained therein using, for example, a suitable adhesive, a friction fit, or other suitable attachment means. In certain embodiments and with reference to FIG. 21C, the handle 370 may comprise a spoked insert 630 disposed within the first bore 380, with the spoked insert 630 comprising a hub 640 extending co-axially through the first bore 380 and defining the second bore 620.

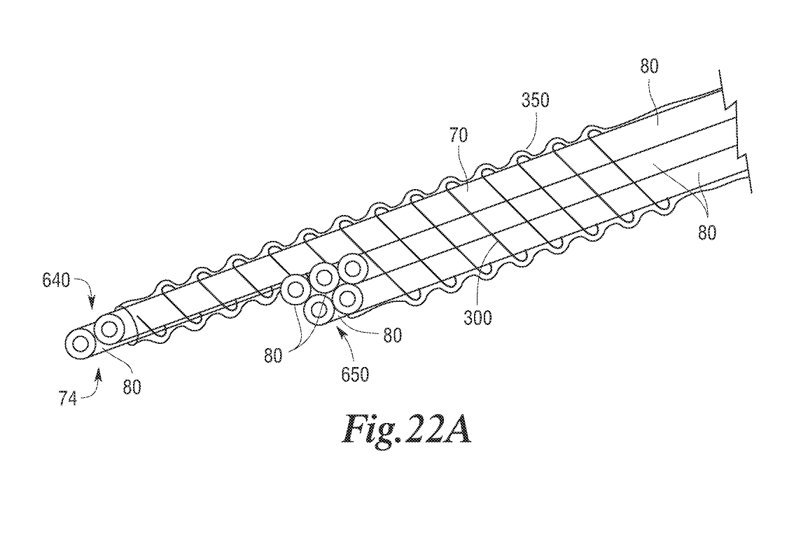

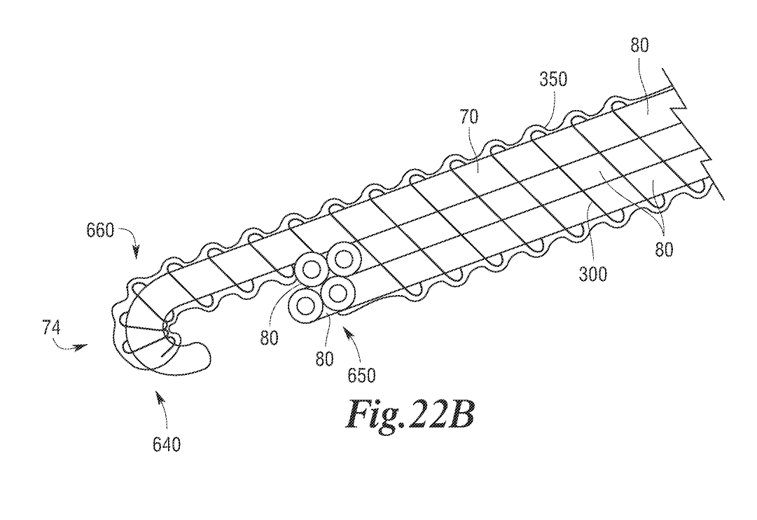

As shown in FIG. 15A, the inner sheath may comprise a first working channel exit site 640 (i.e., a location at which one or more working channels 80 are no longer retained or bundled within the inner sheath 70) distally positioned with respect to the handle 370. In the embodiment of FIG. 15A, the first working channel exit site 640 is adjacent the distal end 74 of the inner sheath 70. According to various embodiments, in addition to a first working channel exit site 640, the inner sheath 70 may comprise a second working channel exit site. As shown in FIG. 22A, for example, the inner sheath 70 may comprise a second working channel exit site 650 positioned between the proximal and distal ends 72, 74 of the inner sheath 70. In such embodiments, a distal end of at least one working channel 80 may be adjacent the first working channel exit site 640 at the distal end 74 of the inner sheath 70, with the distal ends of the remaining working channels 80 being adjacent the second working channel exit site 650. In this way, endoscopic tools may be simultaneously introduced to a treatment site from different locations over the length of the inner sheath 70. A particular advantage of this configuration is the ability to visualize an endoscopic procedure from different perspectives. For example, in one embodiment, one or more working channels 80 adjacent the first working channel exit site 640 may be configured to accommodate one or more imaging devices (e.g., cameras, optics), with the remaining working channels 80 adjacent the second working channel exit site 650 configured to accommodate one or more surgical instruments (e.g., hole-forming devices, graspers, clip appliers, loops, Radio Frequency (RF) ablation devices, harmonic ablation devices, scissors, knives, suturing devices) for performing an endoscopic procedure. By virtue of this arrangement, surgical instruments may be introduced at a location independent of the one or more imaging devices, thus permitting visualization of an endoscopic procedure from a number of different perspectives. Additionally, in certain embodiments, the inner sheath 70 may comprise an articulation joint 660 disposed between the first and second working channel exit sites 640, 650 such that first working channel exit site 640 (and, thus, one or more associated imaging devices) can be variably positioned relative to the second working channel exit site 650. In certain of these embodiments and as shown in FIG. 22B, for example, the articulation joint 660 may be configured such that the first working channel exit site 640 is positionable opposite or substantially opposite the second working channel exit site 650. Accordingly, one or more imaging devices positioned at the first working channel exit site 640 are thus able to provide a “surgical” view of an endoscopic procedure performed using surgical instruments positioned at the second working channel exit site 650. The ability to provide such enhanced visualization represents a significant improvement compared to the restricted field of vision to which conventional endoscopic instruments are often limited.

The particular arrangements of the first and second working channel exit sites 640, 650 in FIGS. 22A and 22B are shown by way of example only, and it will be appreciated that the number and configuration of working channels 80 at each site 640, 650, as well as the number of working channel exit sites, may be varied as necessary. For example, in one embodiment, working channels 80 adjacent the first working channel exit site 640 may be configured to accommodate surgical instruments, while working channels 80 adjacent the second working channel exit site 650 may be configured to accommodate imaging devices. Alternatively, working channels 80 adjacent each exit site 640, 650 may be configured to accommodate imaging devices and surgical instruments simultaneously. In one such embodiment, for example, an endoscopic procedure may be performed using surgical instruments introduced via the second working channel exit site 650, and the procedure may be visualized as needed by alternating imaging devices between the first and second working channel exit sites 640, 650. In another embodiment, surgical tools introduced simultaneously via both exit sites 640, 650 may cooperatively interact to perform an endoscopic procedure (e.g., push/pull tissue separation) when the first working channel exit site 640 is in an articulated position.

According to various embodiments, the plurality of working channels 80 of the inner sheath 70 may comprise flexible tubes constructed from bio-compatible plastics, polymers, or other suitable materials using, for example, extrusion manufacturing processes. In such embodiments, the working channels 80 are sufficiently flexible to permit individual or collective articulation of the working channels 80 about the longitudinal axis L of the inner sheath assembly 50 as necessary, and the cross-sectional rigidity of the working channels 80 is such that each working channel 80 is generally self-supporting and has relatively constant inner and outer diameters. In certain embodiments, the working channels 80 may comprise features such as, for example, spiral wire supports formed in working channel walls to enhance radial support, as well as kink and compression resistance. For example, the working channels 80 may be endoscopic wire-reinforced working channels available from International Polymer Engineering, Tempe, Ariz.

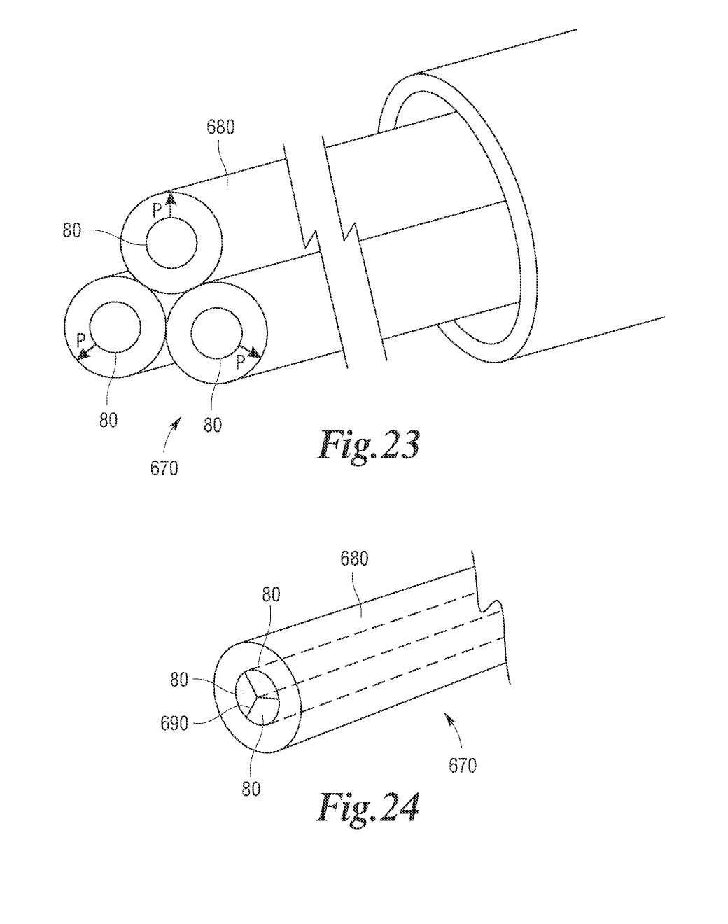

As an alternative to self-supporting working channels 80 of constant diameter, certain embodiments may comprise an inner sheath 70 having a first length 670 including at least one chamber inflatable to define one or more working channels 80. FIG. 23, for example, illustrates an inner sheath 70 having a first length 670 including a chamber 680 inflatable to define three working channels 80. In certain embodiments, the first length 670 may comprise the entire length of the inner sheath 70, while in other embodiments the first length 670 may be less than the entire length of the inner sheath 70.

In certain embodiments, the first length 670 of the inner sheath 70 may be constructed from inflatable tubes interconnected at one or more points over their lengths to define a single chamber 680, for example. In other embodiments, the first length 670 of the inner sheath 70 may comprise multiple chambers 680. In one embodiment, for example, two or more inflatable tubes may define two or more separately inflatable chambers 680. In such embodiments, the number of working channels 80 may be varied by selectively inflating the chambers 680 as necessary.

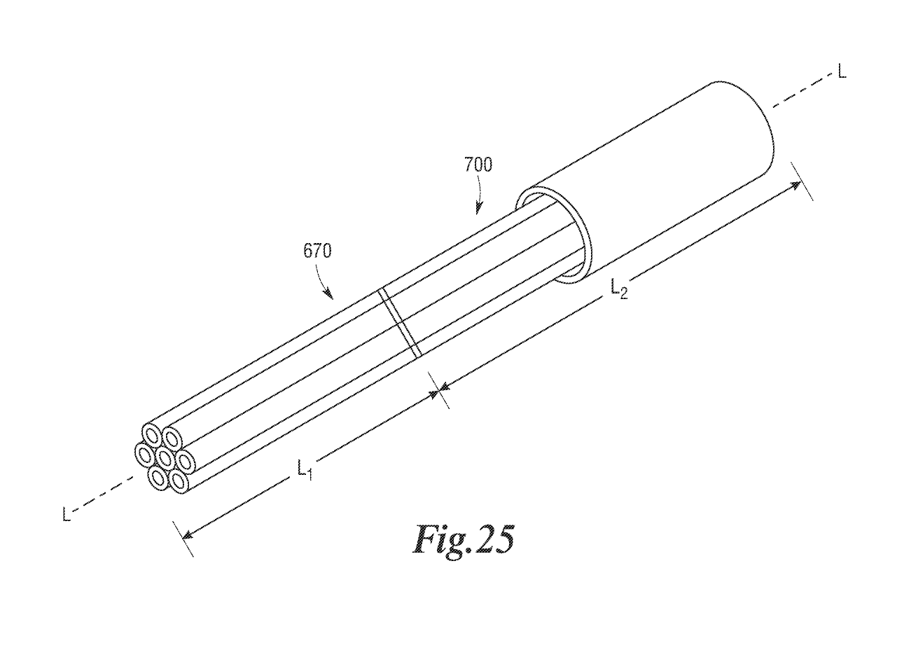

Additionally or alternatively, the first length 670 of the inner sheath 70 may comprise at least one partition 690, with each partition 690 expandable to define at least two working channels 80 when the at least one chamber 680 is inflated. For example, as shown in FIG. 24, an inflatable tube defining a chamber 680 of the first length 670 may contain a partition 690 connected to its inner diameter and extending over a length of the tube. The partition 690 may be constructed from a material of suitable flexibility and strength (e.g., a plastic or polymer) and be configured such that expansion of the corresponding inner diameter of the tube upon its inflation results in a corresponding expansion of the partition 690. Accordingly, the inner diameter of the inflated tube will be divided into at least two working channels 80 depending upon the particular configuration of the partition 690. In the embodiment of FIG. 24, for example, the partition 690 is configured to define three working channels 80.

According to various embodiments, at least a portion of the first length 670 may comprise an elastic material to vary a cross-sectional area of the one or more working channels 80 based on an inflation pressure of the chamber(s) 680. In certain embodiments, for example, at least one chamber 680 may comprise one or more materials of varying elasticity such that the size of the chamber 680 is alterable based on its inflation pressure. In embodiments in which a chamber 680 is implemented using an inflatable tube, for example, the outer diameter of the tube may comprise a material that is relatively elastic such that the inflation pressure may be changed to vary the outer diameter of the tube. In other embodiments, for example, the outer diameter of the tube may comprise a material that is relatively inelastic compared to that of the inner diameter. In such embodiments, the inflation pressure may be varied over a range to change the inner diameter of the tube while maintaining the outer diameter relatively constant.

In certain embodiments, the inner sheath 70 may comprise a second length that is non-inflatable and positioned adjacent the first length 670. In one embodiment and as shown in FIG. 25, for example, a second length 700 may be proximally positioned relative to the first length 670 and comprise one or more working channels 80 to correspondingly communicate with the one or more working channels 80 of the first length when one or more chambers 680 of the first length 670 are inflated. The working channels 80 of the second length 700 may be, for example, self-supporting working channels 80 with relatively constant inner and outer diameters as described above. In this way, inflation and deflation of the one or more chambers 680 of the first length 670 of the inner sheath 70 will have no effect on the more proximal second length 700. Accordingly, injuries that might otherwise result from inflation of the entire length of the inner sheath 70 within a body lumen may be avoided.

Although the second length 700 is shown in the exemplary embodiment of FIG. 25 as being proximally positioned relative to the first length 670, it will be appreciated that the second length 700 may be distally positioned relative to the first length 670 in other embodiments.

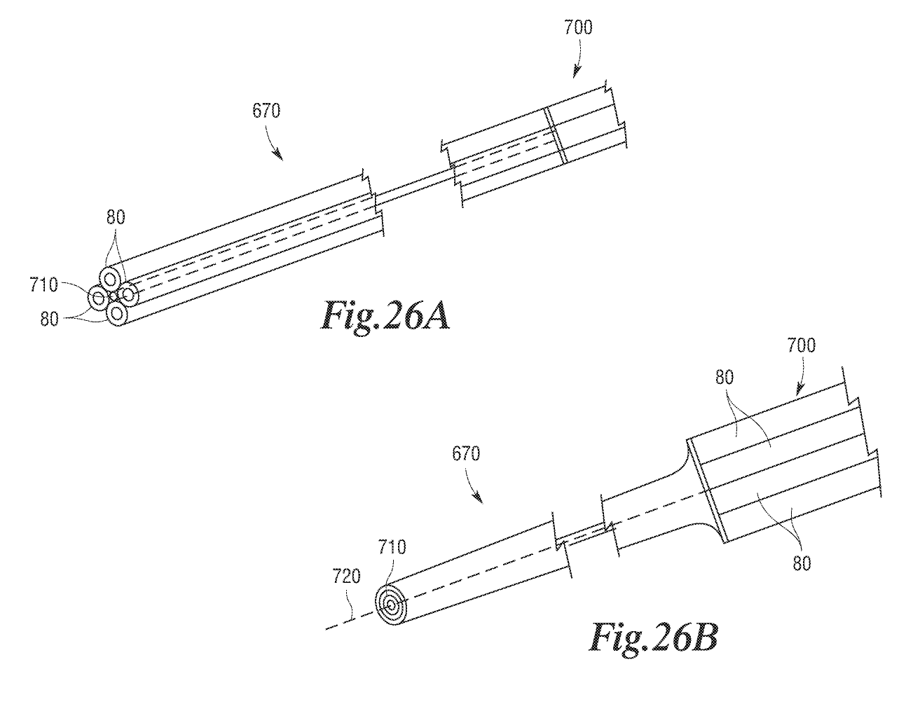

According to various embodiments and as shown in FIG. 26A, the first length 670 of the inner sheath 70 may comprise a guidewire channel 710 to slidably receive a guidewire. In this way, the first length 670 of the inner sheath 70 may be deployed in a deflated state via a guidewire previously inserted through an outer sheath 12, for example. The guidewire channel 710 may extend over the entire first length 670 of the inner sheath 70 and comprise, for example, a plastic tube (e.g., a polyethylene tube) having a flexibility suitable for conforming to tortuous contours of a guidewire, while at the same time having sufficient column strength so that the distal end of the guidewire channel 710 may be advanced over a guidewire by pushing portions of the guidewire channel 710 into the proximal end of the outer sheath 12. In certain embodiments and as shown in FIG. 26B, to enhance passage of the first length 670 of the inner sheath 70 over a guidewire 720 in a deflated state, portions of the inner sheath 70 (e.g., the one or more deflated chambers 680) may be folded and/or wrapped around the guidewire channel 710 such that the cross-sectional profile of the first length 670 is minimized or reduced. In certain embodiments, the guidewire channel 710 may be integrally formed with the first length 670 of the inner sheath 70 and remain in place after the first length 670 is deployed and the guidewire 720 is withdrawn. In other embodiments, the guidewire channel 710 may be removably attached to the first length 670 of the inner sheath 70 (e.g., by virtue of folding or wrapping portions of the inner sheath 70 around the guidewire channel 710). In such embodiments, the first length 670, once deployed, is caused to be released (e.g., as a result of chamber 680 inflation) from the guidewire channel 710 such that both the guidewire 720 and the guidewire channel 710 may be withdrawn.

Although the use of one or more inflatable chambers 680 is described above in connection with working channels 80 of the inner sheath 70, it will also be appreciated that inflatable chambers may also be used to define an outer conduit similar to the outer sheath 12 through which working channels 80 (either self-supporting working channels or working channels defined by inflatable chambers) may be inserted.

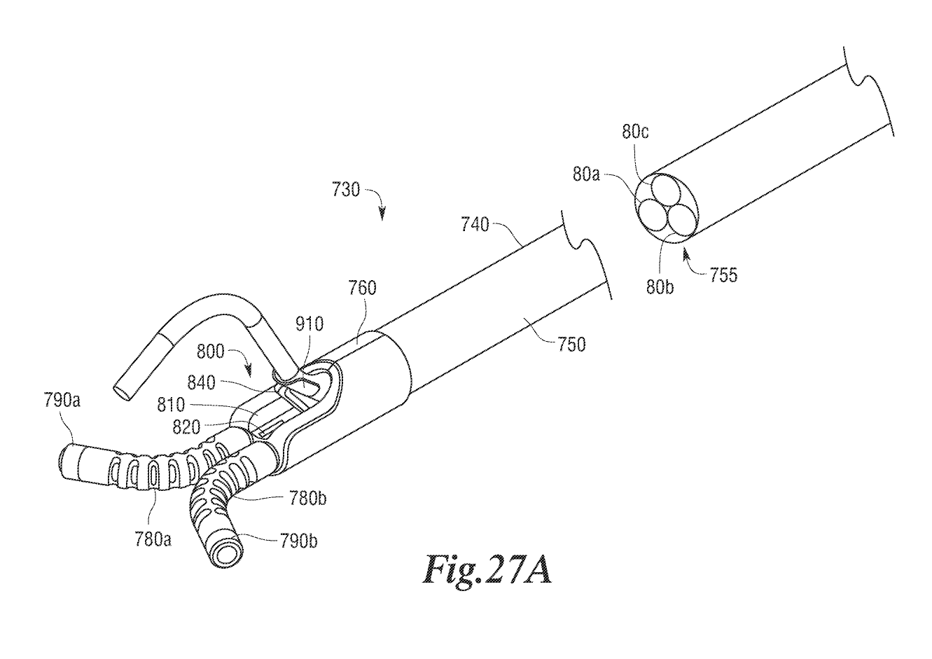

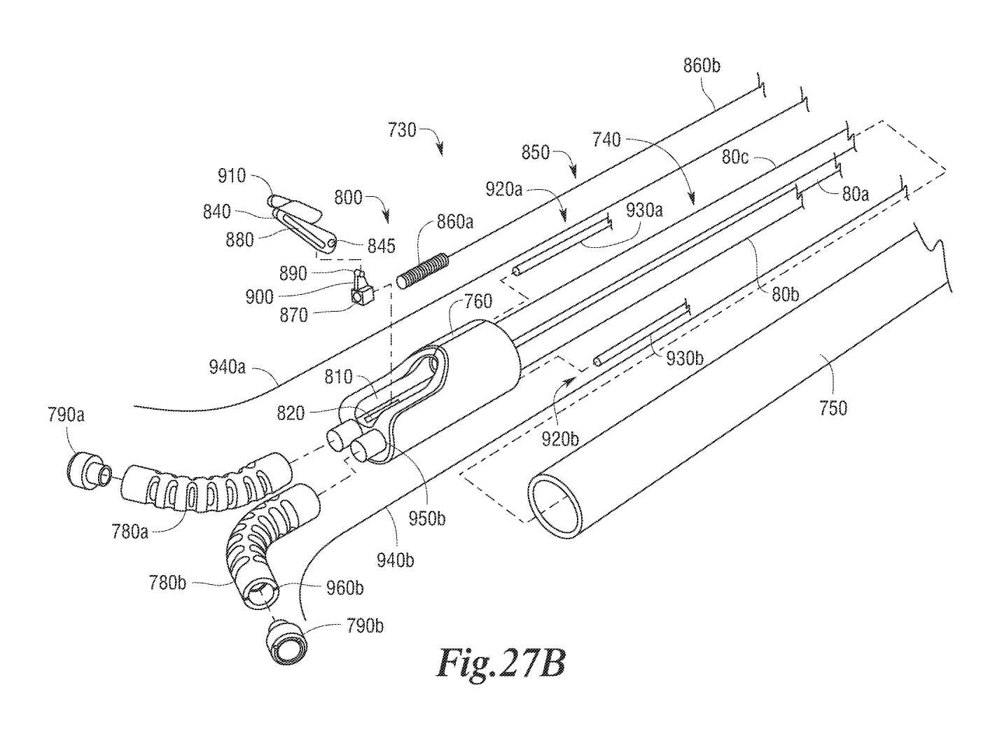

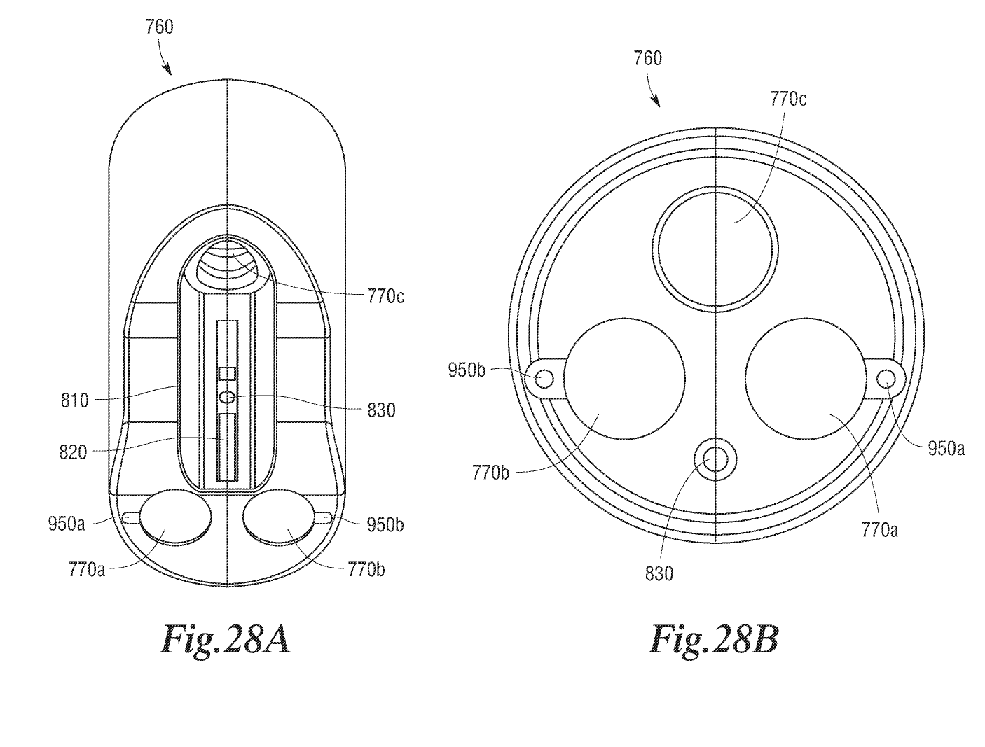

FIGS. 27A and 27B illustrate an assembled view and an exploded view, respectively, of an inner sheath assembly 730 according to another embodiment. As shown, the inner sheath assembly 730 comprises an inner sheath 740 including a plurality of working channels bundled over a common portion of their respective lengths by a flexible sleeve 750 to define a honeycombed cross-sectional area 755. Although the inner sheath 740 is depicted as comprising three working channels 80a, 80b, 80c, it will be appreciated that the number of working channels may generally be two or more. In certain embodiments, the inner sheath 740 and the flexible sleeve 750 may be similar or identical to the inner sheath 70 and the flexible sleeve 350 described above in connection with FIG. 15A. The inner sheath assembly 730 may further comprise a housing 760 defining bores 770a, 770b, 770c (FIGS. 28A and 28B) extending longitudinally and at least partially through the housing 760, with the bores 770a, 770b, 770c receiving distal ends of the working channels 80a, 80b, 80c, respectively, at least partially therethough. As shown in FIG. 27B, for example, the distal ends of the working channels 80a, 80b may be respectively received through the bores 770a, 770b such that distal portions of the working channels 80a, 80b coextend from a distal face of the housing 760, and the distal end of the working channel 80c may be received partially through the bore 770c and terminate within the housing 760 proximal the distal ends of the workings channels 80a, 80b. Flexible articulation joints 780a, 780b may respectively attach to distal ends of the working channels 80a, 80b, and distal tips 790a, 790b may respectively attach to the distal ends of the articulation joints 780a, 780b. In certain embodiments and as discussed in further detail below, the inner sheath assembly 730 may comprise a first actuator 800 to selectively position a distal end of an endoscopic tool (e.g., camera 240, a light) introduced through the working channel 80c, and/or one or more second actuators to manipulate the articulation joints 780a, 780b such that distal ends of endoscopic tools introduced therethrough may be selectively positioned.

FIGS. 28A and 28B illustrate a front perspective view and a rear view, respectively, of the housing 760. The housing 760 may be fabricated from a suitable biocompatible metal or plastic, for example, and, in addition to bores 770a, 770b, 770c, may define a recess 810 in communication with the bore 770c and generally aligned therewith. The recess 810 may be suitably dimensioned to receive and to guide a distal end of an endoscopic instrument introduced through the bore 770c via the working channel 80c and to accommodate components of the first actuator 800. As shown in FIG. 28A, for example, the recess 810 may be generally U-shaped when viewed from the distal end of the housing 760, with a proximal end of the recess 810 transitioning into the distal end of the bore 770c, and with a distal end of the recess 810 opening from the distal face of the housing 760. The housing 760 may further define a slot 820 in communication with a base of the recess 810 and generally aligned therewith to accommodate components of the first actuator 800, and a bore 830 connecting a proximal face of the slot 820 to a proximal face of the housing 760.

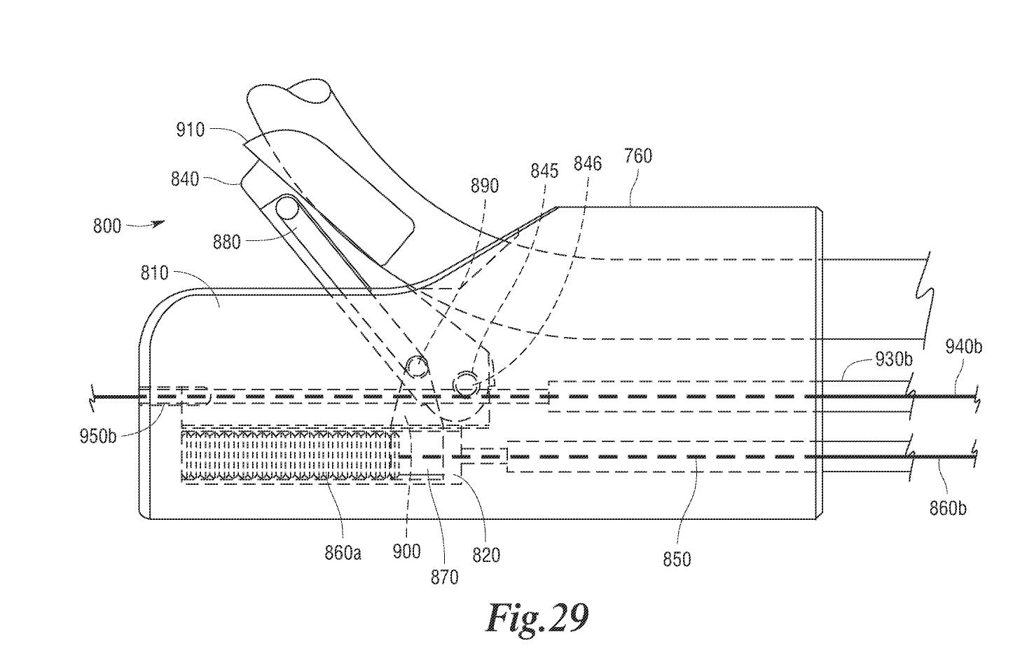

FIG. 29 illustrates a side view of the housing 760 with components of the first actuator 800 installed in the recess 810 and the slot 820. The first actuator 800 may comprise a pivot arm 840 having a proximal end pivotally attached to the housing 760 adjacent a proximal end of the slot 820. In one embodiment, pivotal cooperation between the pivot arm 840 and the housing 760 is accomplished using pivot pins 845 formed on opposing lateral surfaces of the proximal end of the pivot arm 840 that are cooperatively engaged by corresponding pivot recesses 846 defined by opposing lateral surfaces of the proximal end of the slot 820. Accordingly, the pivot arm 840 is pivotable between a lowered, non-deployed position in which the pivot arm 840 is predominantly or entirely contained within the recess 810, and an elevated, deployed position (as shown in FIG. 29) in which at least a distal portion of the pivot arm 840 is pivotably elevated to extend from the recess 810, thereby flexing the distal end of the endoscopic instrument to alter its position.

In certain embodiments, the first actuator 800 may comprise a drive shaft 850 having a distal end 860a disposed in and extending through the slot 820, with the distal end 860a coupled to the pivot arm 840 via a linkage 870 that is slidably disposed in the slot 820. As shown in FIG. 29, at least a portion of the distal end 860a of the drive shaft 850 contained within the slot 820 may be threaded. The linkage 870 may define a bore adapted to threadably receive the distal end 860a of the drive shaft 850. In this way, rotation of the distal end 860a of the drive shaft 850 may be employed to cause translation of the linkage 870 along a length of the slot 820. For example, rotation of the distal end 860a of the drive shaft 850 in a clockwise direction (e.g., as viewed from the proximal end of the inner sheath assembly 730) may cause translation of the linkage 870 in a proximal direction relative to the slot 820, while rotation of the distal end 860a of the drive shaft 850 in an opposite direction may cause the linkage 870 to translate in a distal direction relative to the slot 820. Rotation of the distal end 860a of the drive shaft 850 in this manner may be accomplished by rotating a proximal end 860b of the drive shaft 850 that proximally extends from the bore 830 and through the inner sheath 740. The proximal end 860b of the drive shaft 850 may be connected to a control device (e.g., a motor, a manually rotatable knob) (not shown) for suitably controlling the rotational position of the proximal end 860b, and thus, the translatory position of the linkage 870 relative to the slot 820. In certain embodiments, at least a portion of the proximal end 860b of the drive shaft 850 (e.g., a portion of the drive shaft 850 extending through the inner sheath 740) may be rotatably housed within a flexible sleeve.

As further shown in the embodiment of FIG. 29, the pivot arm 840 may comprise a track 880 in the form of an elongate slot 880 that is defined by lateral surfaces of the pivot arm 840 and that is slidably engaged by a pin 890 formed on an upwardly-extending arm 900 of the linkage 870. The configuration of the slot 880 may be such that when the linkage 870 is translated into its distal-most position relative to the slot 820 (e.g., by suitable rotation of the drive shaft 850), the resulting sliding engagement of the slot 880 by the pin 890 causes the pivot arm 840 to assume its lowered, non-deployed position. Conversely, as the linkage 870 is translated from its distal-most position in a proximal direction, the resulting sliding engagement of the slot 880 by the pin 890 causes the progressive elevation of the pivot arm 840, with the elevated, fully-deployed position of the pivot arm 840 corresponding to the proximal-most position of the linkage 870 relative to the slot 880. In this way, rotation of the drive shaft 850 may be used to selectively adjust the position of the pivot arm 840 between its lowered and elevated positions.

In certain embodiments, the distal end of the pivot arm 840 may comprise a guide surface 910 for slidably contacting a distal end of an endoscopic instrument introduced through the bore 770c via the working channel 80c in order to effectively transfer pivotal movement of the pivot arm 840 to the distal end of the endoscopic instrument. As shown in FIG. 27B, for example, the guide surface 910 may be trough-shaped and comprise a curvature generally matching a curvature of an outer surface of the endoscopic instrument. In this way, the guide surface 910 may conform to a degree to the outer surface of the endoscopic instrument such that the endoscopic instrument is laterally retained on the guide surface 910 while permitting sliding contact of the endoscopic instrument with the guide surface 910 in the distal and proximal directions. In certain embodiments, the guide surface 910 may comprise a lubricious coating (e.g., a biocompatible Teflon® coating) to reduce frictional forces between the guide surface 910 and the endoscopic instrument.

It will be appreciated that translatory control of the linkage 870 may be achieved in a number of ways that do not require a rotatable drive shaft 850. In one embodiment, for example, the first actuator 800 may instead include a control cable assembly (not shown) comprising a flexible guide and a control member slidably disposed therein. A distal end of the flexible guide may be received by and retained within a proximal portion the bore 830 of the housing 760, with a distal end of the control member extending from the distal end of the flexible guide and through a distal portion of the bore 830 to attach to the linkage 870. The flexible guide may proximally extend through a length of the inner sheath 740 and comprise a proximal end attached to, for example, a handle coupled to the inner sheath 740. A distal end of the control member may extend from the proximal end of flexible guide to attach to a suitable mechanical or electromechanical actuator (e.g., a lever actuator, a knob actuator, a trigger actuator, a bar clamp actuator, a syringe grip actuator, a solenoid actuator, a motor actuator) for controllably translating the control member within the guide, thus causing translation of the linkage 870 and concomitant pivotal movement of the pivot arm 840.

In addition to or as an alternative to the use of an active (e.g., movable) actuator (e.g., first actuator 800) to selectively position the distal end of an endoscopic instrument introduced through the working channel 80c, embodiments of the inner sheath assembly 730 may comprise one or more passive (e.g., stationary) guide surfaces to control distal end position by virtue of movement of the distal end relative to the passive guide surface(s). In certain cases, use of passive guide surfaces may be preferable to active actuators in terms of reduced size, ease of manufacture, reduced cost, and/or for addition of components/elements in a space that would otherwise be occupied by components/elements of an active actuator.

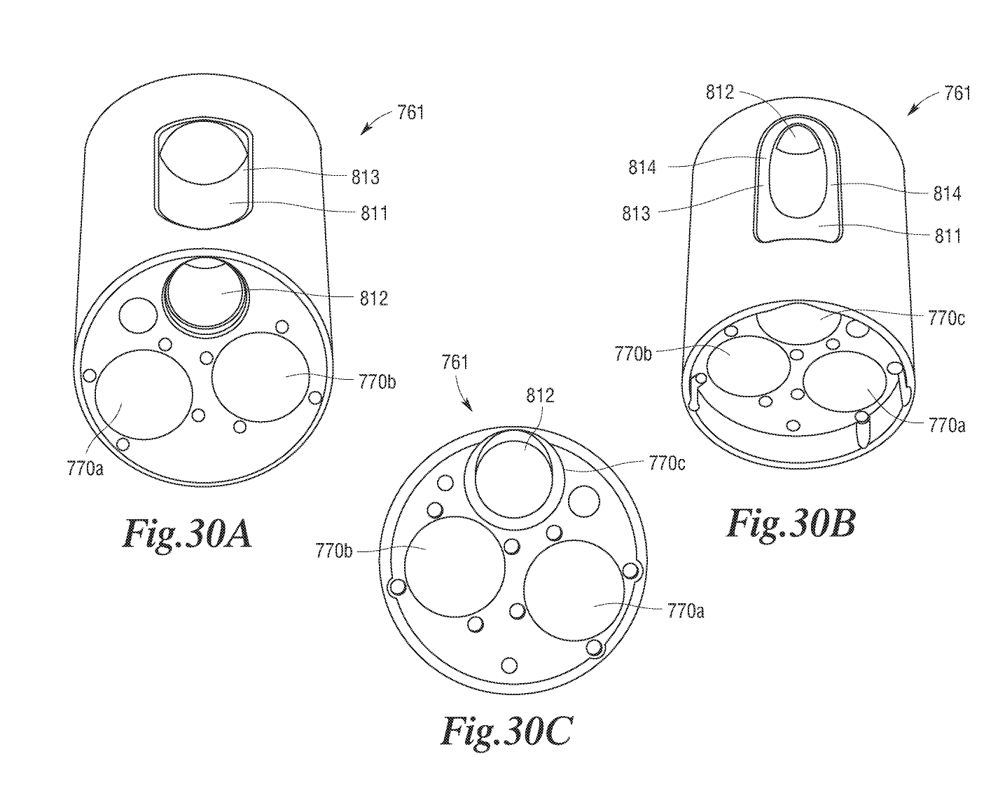

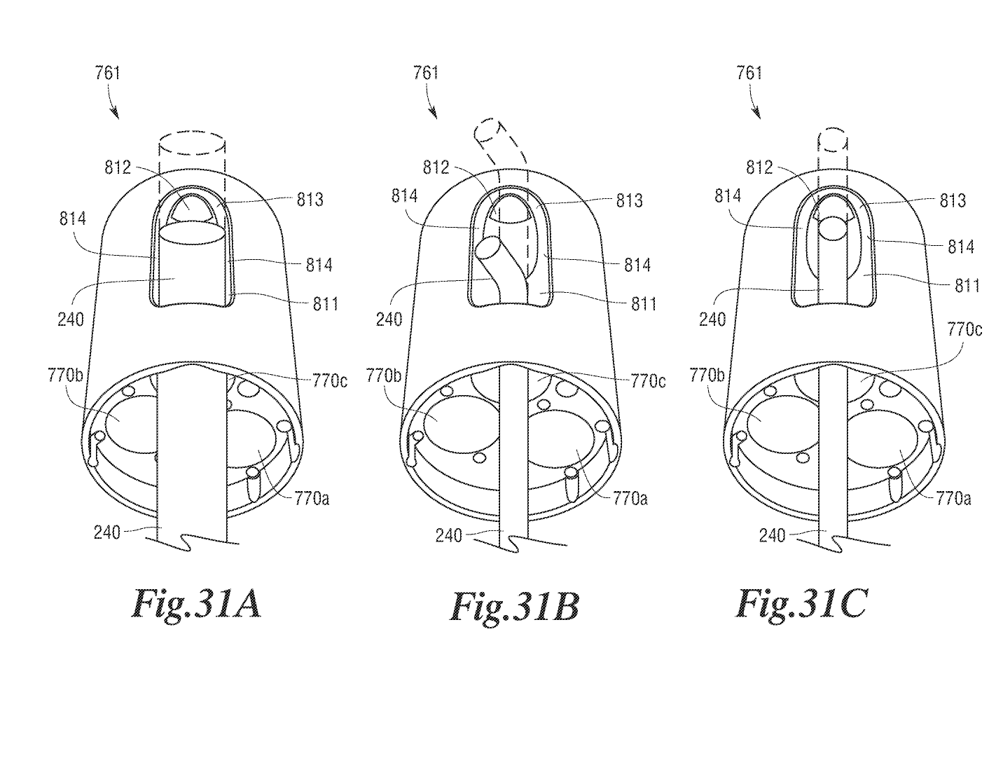

FIGS. 30A and 30B illustrate front perspective and rear perspective views, respectively, of a housing 761 comprising a passive guide surface according to one embodiment. FIG. 30C illustrates a rear view of the housing 761. The housing 761 may be similar in certain respects to the housing 760 and define, for example, bores 770a, 770b and 770c that extend longitudinally and at least partially through the housing 761 and receive the distal ends of the working channels 80a, 80b, 80c, respectively, at least partially therethrough. In FIGS. 30A, 30B and 30C, the working channels 80a, 80b, 8c have been omitted for the sake of clarity. The housing 761 may further define a recess 811 that is in communication with the bore 770c and generally aligned therewith to receive and guide a distal end of an endoscopic instrument introduced through the bore 770c via the working channel 80c. As shown, the housing 761 may define separate openings connected to the recess 811 from which the distal end of the endoscopic instrument may exit the housing 761 subsequent to its introduction into the recess 811 via the bore 770c. For example, a bore 812 may be defined by the housing 761 to provide a transition from the distal end of the recess 811 through the distal face of the housing 761, and an opening 813 may be defined by the housing 761 such that a portion of the recess 811 is exposed through a lateral surface of the housing 761. As shown in FIG. 30B, a distal wall of the recess 811 may define a proximal opening of the bore 812 and comprise a curved surface that is continuous with base and lateral surfaces of the recess 811 and that slopes upward relative to the base surface of the recess 811 in the distal direction. The distal wall of the recess 811 thus defines a ramped guide surface 814 disposed adjacent the proximal opening of the bore 812 to slidably engage and position the distal end of an endoscopic instrument as the distal end is moved in the distal direction relative to the ramped guide surface 814. In certain embodiments, for example, a width of a distal tip portion of the endoscopic instrument (e.g., a distal tip portion of camera 240) may be equal to or slightly smaller than a width of the ramped guide surface 814, but larger than a width of the proximal opening of the bore 812, such that the distal tip portion is not passable through the bore 812. Accordingly, as shown in FIG. 31A, as the distal tip portion of the endoscopic instrument 240 is advanced through the recess 811, the distal tip portion is slidably engaged by the ramped guide surface 814. Continued advancement of the distal tip portion (indicated in FIG. 31A by phantom outline) through the recess 811 causes the distal tip portion to follow the upward-sloping contour of the ramped guide surface 814 and eventually emerge from the recess 811 via the opening 813. In other embodiments, the width of distal tip portion may be smaller than a width of the proximal opening of the bore 812 such that passage of the distal tip portion through either the bore 812 or the opening 813 is possible. In such embodiments and as shown in FIG. 31B, for example, the distal tip portion may be suitably articulated within the recess 811 (e.g., using an actuator of the endoscopic instrument 240) such that at least a portion of the ramped guide surface 814 slidably engages the distal tip portion. Continued advancement of the articulated distal tip portion through the recess 811 (indicated in FIG. 31B by phantom outline) causes the distal tip portion to follow the upward-sloping contour of the ramped guide surface 814 and eventually emerge from the recess 811 via the opening 813. Alternatively, as shown in FIG. 31C, the distal tip portion may be advanced through the recess 811 in an unarticulated state such that distal tip portion is not slidably engaged by the ramped guide surface 814. In this case, continued advancement of the distal tip portion through the recess 811 (indicated in FIG. 31C by phantom outline) results in emergence of distal tip portion from the distal face of the housing 761 via the bore 812.

It will be appreciated that while the housing 761 shown in FIGS. 30A-30C and FIGS. 31A, 31B and 31C defines a single recess 811 with an associated bore 812 and opening 813, it will be appreciated that in other embodiments the housing 761 may define at least one additional recess 811 having an associated bore 812 and opening 813 for selectively positioning the distal end of an endoscopic instrument introduced through other working channel(s). In one such embodiment, for example, the housing 761 may define a recess 811 and an associated bore 812 and opening 813 for each bore 770a, 770b, 770c.

Embodiments of the inner sheath assembly 730 may further comprise one or more second actuators to controllably manipulate the articulation joints 780a, 780b. In one such embodiment, for example, each articulation joint 780a, 780b may be manipulated by a corresponding second actuator 920a, 920b, with each actuator 920a, 920b respectively comprising a flexible guide 930a, 930b and a corresponding control member 940a, 940b slidably disposed therein. As shown in FIGS. 28A, 28B and 29, the housing 760 may define bores 950a, 950b extending longitudinally through the housing 760 between the proximal and distal faces thereof for respectively accommodating distal portions of the second actuators 920a, 920b. Each bore 950a, 950b may define a first diameter to receive and retain a distal portion of the corresponding flexible guide 930a, 930b, and a second diameter distal the first diameter to receive a distal portion of the corresponding control member 940a, 940b. Distal portions of the control members 940a, 940b passed through bores 950a, 950b of the housing 760 may be slidably received through corresponding auxiliary bores 960a, 960b defined by the sidewalls of the articulation joints 780a, 780b, with the auxiliary bores 960a, 960b being respectively aligned with the bores 950a, 950b when the articulation joints 780a, 780b are in an un-articulated state. Distal tips of the control members 940a, 940b may respectively attach to the articulation joints 780a, 780b adjacent the distal ends of their corresponding auxiliary bores 960a, 960b. In this way, each control member 940a, 940b may be slidably translated through its respective flexible guide 930a, 930b, bore 950a, 950b and auxiliary bore 960a, 960b to controllably manipulate the corresponding articulation joint 780a, 780b. In certain embodiments, for example, independent translation of the control members 940a, 940b may be accomplished using a suitable mechanical or electromechanical actuator (e.g., a lever actuator, a knob actuator, a trigger actuator, a bar clamp actuator, a syringe grip actuator, a solenoid actuator, a motor actuator) (not shown) attached to the proximal end of each control member 940a, 940b adjacent a handle coupled to the inner sheath 740.

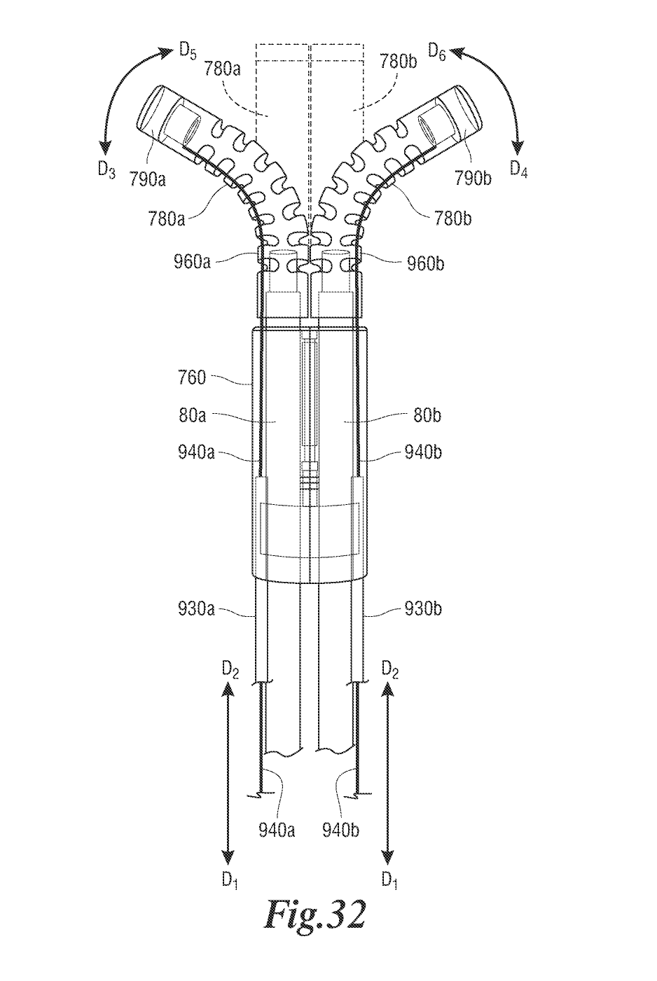

FIG. 32 is a bottom view of a distal portion of the inner sheath assembly 730 illustrating deflection of the articulation joints 780a, 780b in response to translation of their corresponding control members 940a, 940b. As shown, both control members 940a, 940b have been translated equal distances in the proximal direction D₁, thus causing the articulation joints 780a, 780b to be equally deflected in directions D₃ and D₄, respectively. Subsequent translation of the control members 940a, 940b in the distal direction D₂ will reduce the degree of deflection by causing the articulation joints 780a, 780b to move in directions D₅ and D₆, respectively, such that the articulation joints 780a, 780b eventually assume their un-deflected positions (indicated in FIG. 32 by the phantom outline of the articulation joints 780a, 780b). Although not illustrated in FIG. 32, it will be appreciated that the articulation joints 780a, 780b may be deflected in the same direction by translating the control members 940a, 940b in opposite directions. For example, translating control member 940a in the proximal direction D₁ while simultaneously translating control member 940b in the distal direction D₂ will result in the deflection of the articulation joints 780a, 780b in the direction D₃. Conversely, translating control member 940a in the distal direction D₂ while simultaneously translating control member 940b in the proximal direction D₁ will result in the deflection of the articulation joints 780a, 780b in the direction D₄.

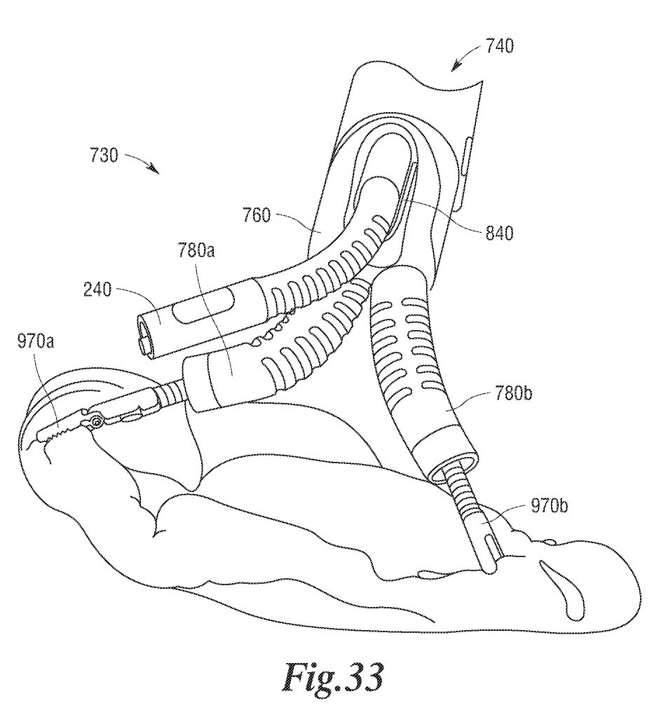

FIG. 33 illustrates a deployment of endoscopic instruments at a treatment site using the inner sheath assembly 730 of FIG. 27A. Although only the distal portion of the inner sheath assembly 730 is shown in FIG. 33, it will be appreciated that a proximal portion of the inner sheath assembly 730 may contained in outer sheath (e.g., the outer sheath 12 of FIG. 1). As shown, graspers 970a, 970b have been introduced to the treatment site via the articulation joints 780a, 780b and attached to corresponding portions of the treatment site. Subsequent manipulation of the articulation joints 780a, 780b in opposite directions has exposed a portion of the treatment site for visualization using a camera 240 introduced to the treatment site via the working channel 80c. The pivot arm 840 is shown in the lowered, non-deployed position.

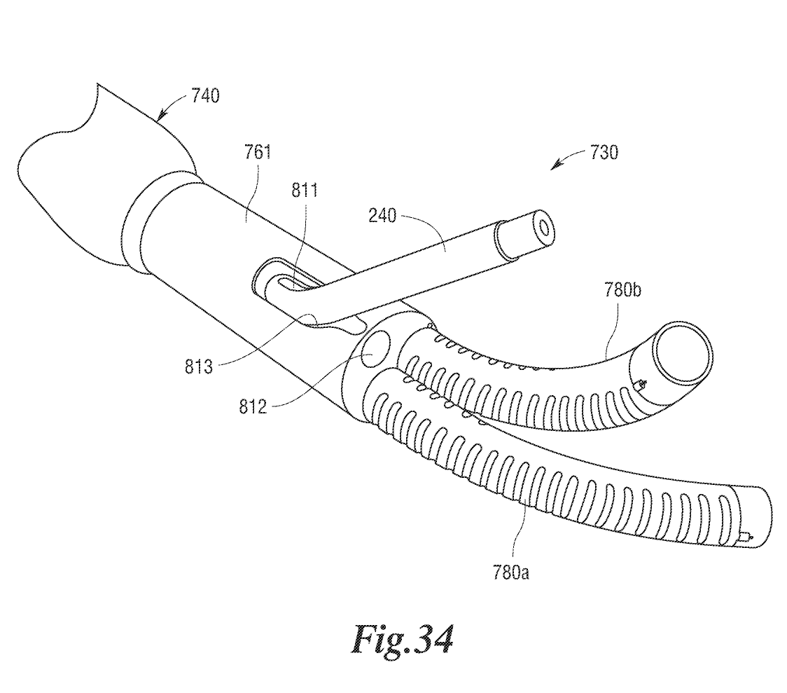

FIG. 34 illustrates the deployment of an endoscopic instrument using an inner sheath assembly 730 comprising the housing 761 of FIGS. 30A, 30B and 30C. The distal end of the endoscopic instrument 240 has been previously advanced into the recess 811 via the bore 770c (not shown) and engaged by the ramped guide surface 814 (not shown), thus causing the distal tip portion of the endoscopic instrument to emerge from the recess 811 via the opening 813 as shown.

While the illustrative embodiments have been described in considerable detail, it is not the intention of the applicant to restrict or in any way limit the scope of the appended claims to such detail. Additional advantages and modifications may readily appear to those skilled in the art. The guide system embodiments represent vast improvements over prior overtube and sheath arrangements. Not only can the system allow the clinician to attain a desired viewing orientation during the operation while maintaining desired insufflation of the area, the guide system also provides the added flexibility for accommodating instrument exchanges, instruments of various sizes and, if necessary, extraction of relatively large portions of tissue therethrough. In addition, the ability to freely move the inner sheath relative to the outer sheath (when unlocked) and also the ability to freely move the endoscopic tools within the inner and outer sheaths provide the clinician with the ability to use such instruments to manipulate and treat tissue as needed.

Furthermore, a variety of different inner sheath configurations may be employed with a single outer sheath/handle assembly arrangement to enable the clinician to perform a variety of different surgical procedures. For example, an inner sheath may have a specific number of appropriately sized working channels that are specifically suited for a particular procedure. The guide system may include several of such inner sheaths, such that the system may be advantageously used to perform several different surgical procedures, simply by using the appropriately configured inner sheath(s).

Those of ordinary skill in the art will also understand that the guide system may effectively employ a variety of different camera arrangements. For example, to further enhance the surgical experience, a camera may be employed that has zoom capability (either digital or optical). Such a camera may be employed to mimic laparoscopic capabilities associated with moving a laparoscope during laparoscopic surgery for example, to provide a stadium view and a detailed view of the tissue as required by the clinician.

While the embodiments have been described, it should be apparent, however, that various modifications, alterations and adaptations to the embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Those of ordinary skill in the art will appreciate that the devices disclosed herein may be provided in a kit that may, for example, be directed to a particular surgical procedure. For example, a kit may include a guide system 10 of the present invention in combination with a disposable endoscope that may or may not have a working channel therein. The guide system 10 may include a steerable outer sheath 12 and handle assembly 20 as well as at least one inner sheath 70 with a working channel configuration that may be particularly well-suited to accommodate those endoscopic tools likely to be employed during a particular surgical procedure. In other embodiments, the kit may include a plurality of inner sheaths 70 that each have different working channel configurations therein. Such kit arrangements provide the clinician with the added flexibility to select the appropriate inner sheath 70 for a particular procedure and to remove and insert other inner sheaths 70 with different working channels that are better suited to accommodate different endoscopic tools as the surgical procedure progresses.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A guide system for accommodating an endoscopic tool, comprising:
a flexible inner sheath comprising a plurality of working channels, wherein the working channels are bundled over a common portion of their respective lengths, and wherein the working channels collectively define a substantially honeycombed cross-sectional area; and
a handle coupled to the inner sheath adjacent a proximal end of the inner sheath.

2. The guide system of claim 1, comprising:
a hollow outer sheath having a proximal end and a distal end, wherein the distal end is substantially steerable, and wherein the inner sheath is sized relative to the hollow outer sheath to permit the inner sheath to be selectively rotated and axially moved with the hollow outer sheath such that a distal end of the inner sheath is selectively protrudable beyond the distal end of the hollow outer sheath.

3. The guide system of claim 1, wherein the cross-sectional area is substantially circular.

4. The guide system of claim 1, wherein the cross-sectional area is substantially non-circular.

5. The guide system of claim 4, wherein the cross-sectional area is clover-shaped.

6. The guide system of claim 1, comprising:
at least one retainer disposed over a length of the inner sheath to retain the working channels in a substantially fixed orientation relative to each other.

7. The guide system of claim 6, wherein the at least one retainer is selected from the group consisting of:
a flexible coil defining a longitudinal bore to receive the plurality of working channels;
a tube comprising a series of slits to make the tube flexible, the tube defining a central opening to receive the plurality of working channels; and
a flexible sleeve defining a longitudinal bore to receive the plurality of working channels.

8. The guide system of claim 7, wherein the at least one retainer comprises the coil.

9. The guide system of claim 8, wherein the at least one retainer comprises the sleeve conformably disposed over the coil.

10. The guide system of claim 7, wherein the at least one retainer comprises the sleeve conformably disposed over the plurality of working channels.

11. The guide system of claim 7, wherein the plurality of working channels is twisted in a direction about a longitudinal axis of the inner sheath.

12. The guide system of claim 11, wherein the at least one retainer comprises the coil, and wherein the plurality of working channels is twisted in a direction opposite a twist of the coil.

13. The guide system of claim 6, comprising:
at least one first actuator to position a distal end of the inner sheath.

14. The guide system of claim 13, wherein each at least one first actuator comprises:
a flexible guide extending over a length of the inner sheath, wherein the guide comprises a distal end attached to the at least one retainer adjacent a distal end of the at least one retainer, and wherein the guide comprises a proximal end adjacent the handle;
a control member slidably disposed within the guide, wherein the control member comprises a distal end extending from the distal end of the guide and attached to the distal end of the at least one retainer, and wherein the control member comprises a proximal end extending from the proximal end of the guide; and
a control device attached to the proximal end of the control member for slidably translating the control member through the guide to position the distal end of the inner sheath.

15. The guide system of claim 1, comprising:
at least one second actuator to position a distal end of a first working channel relative to a distal end of one or more second working channels.

16. The guide system of claim 15, wherein each at least one second actuator comprises:
a flexible guide extending over a length of the first working channel, wherein the guide comprises a distal end adjacent a distal end of the first working channel, and wherein the guide comprises a proximal end adjacent the handle;
a control member slidably disposed within the guide, wherein the control member comprises a distal end extending from the distal end of the guide and attached to the distal end of the first working channel, and wherein the control member comprises a proximal end extending from the proximal end of the guide; and
a control device attached to the proximal end of the control member for slidably translating the control member through the guide to position the distal end of the first working channel relative to a distal end of the one or more second working channels.

17. The guide system of claim 1, comprising:
a tip disposed over a distal end of the inner sheath.

18. The guide system of claim 1, comprising:
a flexible core coupled to the handle and extending distally from the handle, wherein at least a portion of each working channel is wrapped around the core to at least partially transfer torque applied to the handle to the plurality of working channels via the core.

19. The guide system of claim 18, wherein the core comprises one or more of a solid shaft, a cable, and a tube.

20. The guide system of claim 1, wherein the inner sheath comprises a first working channel exit site and a second working channel exit site, wherein the first and second working channel exit sites are distally positioned with respect to the handle, wherein the first working channel exit site is positioned at a distal end of the inner sheath, and wherein the second working channel exit site is positioned between the distal and proximal ends of the inner sheath.

21. The guide system of claim 20, wherein a distal end of at least one working channel is adjacent the first working channel exit site, and wherein distal ends of the remaining working channels are adjacent the second working channel exit site.

22. The guide system of claim 20, wherein the inner sheath is articulatable between the first and second working channel exit sites to position the first working channel exit site relative to the second working channel exit site.

23. The guide system of claim 22, wherein the first working channel exit site is positionable substantially opposite the second working channel exit site.

24. A surgical device, comprising:
a flexible elongated inner sheath to be received through a body lumen, wherein the inner sheath comprises a first length having at least one chamber inflatable to define one or more working channels.

25. The surgical device of claim 24, wherein the inner sheath comprises a second length adjacent the first length, wherein the second length comprises one or more working channels to correspondingly communicate with the one or more working channels of the first length when the at least one chamber is inflated, and wherein the second length is non-inflatable.

26. The surgical device of claim 24, wherein at least a portion of the first length comprises an elastic material to vary the cross-sectional area of the one or more working channels based on an inflation pressure of the at least one chamber.

27. The surgical device of claim 24, wherein the first length comprises a partition expandable to define at least two working channels when the at least one chamber is inflated.

28. The surgical device of claim 24, wherein the inner sheath comprises a guidewire channel to slidably receive a guidewire.

29. The surgical device of claim 24, wherein the at least one chamber is inflatable to define a plurality of working channels, and wherein the plurality of working channels collectively define a substantially honeycombed cross-sectional area.
